(19) United States
(12) Patent Application Publication
(10) Pub. No.: US 2023/0414738 A1
(43) Pub. Date: Dec. 28, 2023

(54) HAEMOPHILUS INFLUENZAE VACCINE AND METHODS OF USE

(71) Applicant: THE ROCHESTER GENERAL HOSPITAL, Rochester, NY (US)

(72) Inventor: Michael Pichichero, Rochester, NY (US)

(21) Appl. No.: 18/043,241

(22) PCT Filed: Sep. 10, 2021
(86) PCT No.: PCT/US21/49765

§ 371 Date: Feb. 27, 2023

Related U.S. Application Data
(60) Provisional application No. 63/076,461, filed on Sep. 10, 2020.

(51) Int. Cl. A61K 39/102; C07K 14/285; A61P 31/04; C12N 15/70

(52) U.S. Cl. CPC A61K 39/102; C07K 14/285; C12N 2800/101; C12N 15/70; C07K 2319/00; A61P 31/04

(57) ABSTRACT

The present disclosure is directed to a fusion protein comprising all or part of two or more Haemophilus influenzae (Hi) proteins selected from the group consisting of Omp26, P6, P4, PD and PF, wherein at least one of the Hi proteins thereof comprises a lipid moiety, and vaccines and immunogenic compositions comprising such fusion proteins. Methods of treating or preventing a disorder associated with an Hi infection in a subject are also provided.

### CROSS REFERENCE TO RELATED APPLICATIONS

This application claims the priority benefit of U.S. Provisional Patent Application No. 63/076,461, filed Sep. 10, 2020, hereby incorporated by reference in its entirety.

### FIELD OF THE INVENTION

The present disclosure is in the field of Haemophilus influenzae vaccines.

### INCORPORATION BY REFERENCE OF SEQUENCE LISTING

This application contains a Sequence Listing in computer readable form (filename: 55894PCT_SeqListing.txt; 83,018 bytes—ASCII text file; created Sep. 8, 2021), which is incorporated herein by reference in its entirety and forms part of the disclosure.

### BACKGROUND

Haemophilus influenzae (Hi) is a gram-negative coccobacillus and a strict human commensal bacteria. Strains of Hi are either encapsulated in a polysaccharide capsule or are non-encapsulated and are accordingly classified into typeable (encapsulated) and non-typeable (non-encapsulated) strains.

Polysaccharide encapsulated Hi are of serotypes a, b, c, d, e and f. Hi encapsulated type b was a dominant cause of invasive Hi infections prior to the invention of a Hib vaccine. Hib vaccines are not available world-wide due to costs and administration challenges. Invasive Hi expressing capsule types a, c, d, e and f have been emerging in recent years. Non-typeable Haemophilus influenzae (Hi) represents the majority of the colonizing strains and, although rarely invasive, are responsible for a significant proportion of mucosal disease including otitis media, sinusitis, chronic conjunctivitis and chronic or exacerbation of lower respiratory tract infections. Currently, approximately 30%, and as much as 62% of Hi are resistant to penicillins. Carriage is estimated at 44% in children and approximately 5% in adults, and can persist for months. Neither the pathogenic mechanisms nor the host immunological response has been fully defined for otitis media caused by Hi.

Otitis media is a common disease in children less than 2 years of age. It is defined by the presence of fluid in the middle ear accompanied by a sign of acute local or systemic illness. Acute signs include ear pain, ear drainage, and hearing loss, whereas systemic signs include fever, lethargy, irritability, anorexia, vomiting or diarrhea. Streptococcus pneumoniae and Hi are the most predominant bacteria that cause the condition, accounting for 25-50%, and 15-30% of the species cultured, respectively. In addition, Hi is responsible for 53% of recurrent otitis media. Approximately 60% and 80% of children have at least one episode of the disease by 1 and 3 years of age respectively (the peak being around 10 months).

Nontypeable Hi is the most common cause of acute exacerbation of chronic bronchitis (AECB), and the presence of a new strain of Hi from the sputum of a patient with chronic bronchitis increases the relative risk of an exacerbation twofold (Sethi et al., N. Engl. J. Med., 347:464-471, 2002).

Bacterial infections (including Hi infections) has also been found to be associated with chronic obstructive pulmonary disorder (COPD). (Albertson et al., J. Am. Geriat. Soc., 58:570-579, 2010). The syndrome of COPD consists of chronic bronchitis (CB), bronchiectasis, emphysema, and reversible airway disease that combine uniquely in an individual patient. Older patients are at risk for COPD and its components—emphysema, chronic bronchitis (CB), and bronchiectasis. Bacterial and viral infections play a role in acute exacerbations of COPD (AECOPD) and in acute exacerbations of CB (AECB) without features of COPD. Older patients are at risk for bacterial infections (including nontypeable Hi infections) during their episodes of AECOPD and AECB.

CB is a progressive disease characterized by chronic sputum production and defined by at least 3 months of cough and sputum in each of 2 consecutive years after the elimination of tuberculosis, lung cancer, and other causes of cough (Balter et al., Can. Respir. J. 10, 3B-32B, 2003; Bronton et al., Am. J. Manage Care, 10:689-696, 2004). CB is reported in the majority of patients with COPD (Balter, supra) AECB causes recurrent attacks in these patients associated with worsening bronchial inflammation.

There is evidence that protective immunity does exist for Hi, however antigenic drift in the epitopes naturally involved in infection (outer-membrane proteins P2, P4, P6) plays a major role in the ability of Hi to evade the immune defense of the host.

### SUMMARY OF THE INVENTION

In one aspect, described herein is a fusion protein comprising all or part of two or more Haemophilus influenzae (Hi) proteins selected from the group consisting of Omp26, P6, P4, PD and PF, wherein at least one of the Hi proteins are lipidated. In some embodiments, the fusion protein comprises Omp26 and P6, wherein the Omp26 protein is lipidated. In some embodiments, the fusion protein comprises Omp26 and P6, wherein the P6 protein is lipidated. In some embodiments, the fusion protein comprises two lipidated Hi proteins.

In various embodiments the construct is LOmp26φP6 (SEQ ID NO: 31), LP4ϕOmp26 (SEQ ID NO: 29), L-P6φpNL-PD (SEQ ID NO: 39), L-PDφNL-PF (SEQ ID NO: 40), L-PDφNL-P6 (SEQ ID NO: 41), L-P6φNL-PD (SEQ ID NO: 42), L-OMP26φNL-PD (SEQ ID NO: 43), L-PDφNL-OMP26 (SEQ ID NO: 44), or L-PFφNL-P6 (SEQ ID NO: 45). An exemplary sequence is L-PDφNL-PF, signal sequence in bold, linker underlined.

(SEQ ID NO: 40)
MKTTLKMTALAALSAFVLAGCSSHSSNMANTQMKSDKIIIAHRGASGYL
PEHTLESKALAFAQHADYLEQDLAMTKDGRLVVIHDHFLDGLTDVAKKF
PHRHRKDGRYYVIDFTLKEIQSLEMTENFETKDGKQAQVYPNRFPLWKS
HERIHTFEDEIEFIQGLEKSTGKKVGIYPEIKAPWFHHQNGKDIAAETL
KVLKKYGYDKKTDMVYLQTFDFNELKRIKTELLPQMGMDLKLVQLIAYT
DWKETQEKDPKGYWVNYNYDWMFKPGAMAEVVKYADGVGPGWYMLVNKE
ESKPDNIVYTPLVKELAQYNVELHPYTVRKDALPEFFTDVNQMYDTLLN
KSGATGVFTDFPDTGVEFLKGIKGSGGGGKFKVVTTFTVIQDIAQNVAG
DAATVESIKPGAEIHEYEPTPKDIVKAQSADLILWNGLNLERWFERFFQ
NVKDKPAVVVTEGIQPLSIYEGPYKDAPNPHAWMSPSNALIYIENIKNA
LVKYDPQNAAVYEKNAADYAQKIKQLDEPLRAKLAQIPEAQRWLVTSEG
AFSYLAKDYNLKEGYLWPINAEQQGTPQQVRKVIDLVRKNNIPVVFSES
TISAKPAQQVAKESGAKYGGVLYVDSLSAKNGPVPTYIDLLNVTVSTIV
KGFGK

In various embodiments, the fusion protein can comprise a signal sequence selected from the group consisting of SS1: Natural P6 signal sequence, MNKFVKSLLVAGSVAALAAC (SEQ ID NO: 36); SS2: E. coli natural signal sequence of Pal protein, MQLNKVLKGLMIALPVMAIAAC (SEQ ID NO: 37), and SSP4: H. influenzae signal sequence of P4 protein, MKTTLKMTALAALSAFVLAGC (SEQ ID NO: 38). It is contemplated that when the signal sequence is used in the fusion protein sequence, the signal sequence may be cleaved at the C residue and the lipid moiety is attached.

In some embodiments, at least one Hi protein comprises a lipid moiety comprising one or more of C18, C16, C14, C12, and C10 fatty acids. In some embodiments, the at least one Hi protein comprises a lipid moiety selected from a diacyl and/or a triacyl fatty acid, or combinations thereof. In some embodiments, the at least one Hi protein comprises a lipid moiety selected from an N-acylated or O-acylated fatty acid. In some embodiments, the Hi protein comprises a lipid moiety at the N terminus of the protein. In some embodiments, two Hi proteins in the fusion protein are lipidated.

In some embodiments, the fusion protein comprises lipidated Omp26 and non-lipidated P6, wherein the lipid comprises a C16 fatty acid. In some embodiments, the fusion protein comprises lipidated P6 and non-lipidated Omp26, wherein the lipid comprises a C16 fatty acid.

In some embodiments, Omp26 comprises all or part of the amino acid sequence set out in SEQ ID NO: 2. In some embodiments, P6 comprises all or part of the amino acid sequence set out in SEQ ID NO: 4. In some embodiments, the P4 protein comprises the amino acid sequence set forth in SEQ ID NO: 6. In some embodiments, the PD protein comprises the amino acid sequence set forth in SEQ ID NO: 8. In some embodiments, the PF protein comprises the amino acid sequence set forth in SEQ ID NO: 10.

In various embodiments, the fusion protein optionally comprises a linker. In some embodiments, the linker is a peptide linker. In some embodiments, the peptide linker is a Gly-Ser linker. In some embodiments, the peptide linker is GlySerGlyGlyGlyGly (SEQ ID NO: 12).

In some embodiments, the fusion protein comprises the amino acid set out in SEQ ID NOS: 29 or 31.

Vaccines and immunogenic compositions comprising the fusion proteins described herein are also contemplated.

The present disclosure also provides methods for making a fusion protein comprising all or part of two or more Haemophilus influenza (Hi) proteins selected from Omp26, P6, P4, PD and PF, wherein at least one of the Hi proteins comprises a lipid moiety, the method comprising (i) providing a nucleic acid sequence encoding a lipid moiety signal sequence region; (ii) providing a first nucleic acid sequence encoding all or part of Hi protein Omp26, P6, P4, PD or PF; (iii) providing a second nucleic acid sequence encoding all or part of Hi protein Omp26, P6, P4, PD or PF, wherein the second nucleic acid encodes a different Hi protein from (ii); (iv) optionally providing a third or additional nucleic acid sequences encoding all of part of one or more additional Hi proteins Omp26, P6, P4, PD or PF, wherein the third or more nucleic acids encode different Hi proteins of (ii) and (iii); (v) inserting the nucleic acid sequences (i)-(iv) into a plasmid vector capable of expressing the nucleic acids; (vi) transfecting the plasmid vector into a host cell capable of expressing the nucleic acids molecules and expressing the fusion protein; and (vii) purifying the recombinant fusion protein expressed in the host cell. In various embodiments, the host cell is grown in minimal media.

In another embodiment, contemplated herein is a method of making a fusion protein comprising all or part of two or more Haemophilus influenzae (Hi) proteins selected from the group consisting of Omp26, P6, P4, PD and PF, wherein at least one of the Hi proteins is lipidated, the method comprising: i) inserting a nucleic acid encoding a lipid moiety signal sequence region upstream of a first nucleic acid encoding all or part of a

Haemophilus influenzae (Hi) protein Omp26, P6, P4, PD or PF in a plasmid vector; ii) inserting a second nucleic acid encoding all or part of a Haemophilus influenzae (Hi) protein Omp26, P6, P4, PD or PF in the plasmid vector; iii) optionally inserting a third or additional nucleic acid sequences encoding one or more additional Hi proteins or fragment thereof selected from the group consisting of Omp26, P6, P4, PD and PF; iv) transfecting the plasmid vector into a host cell capable of expressing the nucleic acid molecules; v) purifying the fusion protein expressed by the plasmid. In various embodiments, the host cell is grown in minimal media.

In various embodiments, the lipid moiety signal sequence is selected from the group consisting of MNKFVKSLLVAGSVAALAAC (SEQ ID NO: 36), with or without the terminal C residue; MQLNKVLKGLMIALPVMAIAAC (SEQ ID NO: 37), with or without the terminal C residue, MKTTLKMTALAALSAFVLAGC (SEQ ID NO: 38) or MKTTLKMTALAALSAFVLAG (SEQ ID NO: 11). In various embodiments, the lipid moiety signal sequence is a P4 signal sequence MKTTLKMTALAALSAFVLAGC (SEQ ID NO: 38) or MKTTLKMTALAALSAFVLAG (SEQ ID NO: 11).

In another aspect, described herein is a method of making a fusion protein comprising all or part of two or more Haemophilus influenzae (Hi) proteins selected from the group consisting of Omp26, P6, P4, PD and PF, wherein at least one of the Hi proteins is lipidated, the method comprising: i) inserting a nucleic acid encoding a P4 lipid moiety signal sequence region upstream of a first nucleic acid encoding all or part of a Haemophilus influenzae (Hi) protein Omp26, P6, P4, PD or PF in a plasmid vector; ii) inserting a second nucleic acid encoding all or part of a Haemophilus influenzae (Hi) protein Omp26, P6, P4, PD or PF in the plasmid vector; iii) optionally inserting a third or additional nucleic acid sequences encoding one or more additional Hi proteins or fragment thereof selected from the group consisting of Omp26, P6, P4, PD and PF; iv) transfecting the plasmid vector into a host cell capable of expressing the nucleic acid molecules; v) purifying the fusion protein expressed by the plasmid.

In another aspect, described herein is a method of making a fusion protein comprising all or part of two or more Haemophilus influenzae (Hi) proteins selected from the group consisting of Omp26, P6, P4, PD and PF, wherein at least one of the Hi proteins is lipidated, the method comprising: i) providing a nucleic acid encoding a P4 lipid moiety signal sequence region; ii) providing a first nucleic acid encoding all or part of a Hi protein Omp26, P6, P4, PD or PF; iii) providing a second nucleic acid encoding all or part of a Hi protein Omp26, P6, P4, PD or PF, wherein the second nucleic acid encodes a different Hi protein from i); iv) optionally providing a third or additional nucleic acid sequences encoding one or more additional proteins or fragment thereof selected from the group consisting of Omp26, P6, P4, PD and PF; v) inserting the nucleic acid sequences i-iv) into a plasmid vector capable of expressing the nucleic acids; vi) transfecting the plasmid vector of iv) into a host cell capable of expressing the nucleic acid molecules and expressing the fusion protein; and, vii) purifying the recombinant fusion protein expressed in the host cell.

In another aspect, described herein is a method of treating or preventing disease associated with a nontypeable Haemophilus influenza (Hi) infection comprising administering a vaccine or immunogenic composition comprising a fusion protein described herein. Diseases associated with Hi infection include, but are not limited to, otitis media, bronchitis, pneumonia, sinusitis, septicemia, endocarditis, epiglottitis, septic arthritis, meningitis, postpartum and neonatal infections, postpartum and neonatal sepsis, acute and chronic salpingitis, epiglottis, pericarditis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic bronchitis, and exacerbation of underlying lung diseases such as chronic bronchitis (AECB), bronchiectasis, or cystic fibrosis, chronic obstructive pulmonary disorder (COPD), and acute exacerbations of COPD (AECOPD).

In some embodiments, the disease associated with a non-typeable Hi infection is otitis media. In some embodiments, the disease associated with a non-typeable Hi infection is acute exacerbation of chronic bronchitis (AECB). In some embodiments, the disease associated with a non-typeable Hi infection is acute exacerbation of chronic obstructive pulmonary disorder (AECOPD).

Also provided is a composition comprising a fusion protein, vaccine or immunogenic composition described herein for use in treating or preventing disease associated with a nontypeable Haemophilus influenza (Hi) infection. In various embodiments, the disclosure provides for use of a composition comprising a fusion protein, vaccine or immunogenic composition described herein in the preparation of a medicament for use in treating or preventing disease associated with a nontypeable Haemophilus influenza (Hi) infection.

In various embodiments, the vaccine or immunogenic composition reduces or prevents colonization in one or more of the sinus, lungs and ears.

In various embodiments, the vaccine or immunogenic composition comprises an LOmp26φP6 fusion protein or LP6ϕOmp26 fusion protein.

In various embodiments, the vaccine or immunogenic composition is administered orally, intravenously, intramuscularly, intranasally, or subcutaneously. In various embodiments, the vaccine or immunogenic composition is administered intranasally.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawing and/or claims.

### BRIEF DESCRIPTION OF THE FIGURES

FIG. 1 is a graph comparing IgG antibodies titers of protein P6 and Omp26 in its lipidated and non-lipidated form as determined by ELISA.

FIG. 2 depicts bar graphs comparing IgG antibody titers of lipidated and non-lipidated fusion proteins as determined by ELISA.

FIGS. 3A and 3B are bar graphs comparing the Hi burden in the nasal lavage (FIG. 3A) and middle ear lavage (FIG. 3B) of mice vaccinated with 3 doses (10 ug each proteins) of non-lipidated P6, lipidated P6 and alum as control. 4-5 adult mice were used per group in study against P6. p-values were calculated using student t-test between groups after log transforming the CFU count.

FIG. 4 illustrates the type of lipidation found on the different Hi lipidated peptides as measured by HEK-BLUE™ TLR2-TLR1 cell stimulation, expressed as % stimulation. PAM3CSK4 positive control is 100% stimulation. *P<0.0001.

FIG. 5 shows an analysis of signal sequence used to attach lipid to protein as measured by activation of HEK-BLUE™ cell stimulation. P values for the difference in 3 signal sequences at two different concentration using Anova.

FIGS. 6A-6C show the immunogenicity of lipidated fusion protein L-P6ϕNL-PD on immunogenicity in vivo as measured by antibody response to PD protein (FIG. 6A) and P6 protein (FIG. 6B) (p-values calculated between groups using student t-test on log 2 transformed antibody levels). FIG. 6C shows L-OMP26φNL-P6 fusion induces significant increase in antibody to OMP26 and P6. p-values calculated using student t-test between groups.

FIG. 7 shows antibody titer after intranasal - immunization with L-OMP26φNL-P6 fusion in animal model B.

FIGS. 8A-8B illustrate the effects of diacyl moieties compared to triacyl lipid moieties on immunogenicity as measured by antibody production (FIG. 8A) or cytokine induction, e.g., CCLS (FIG. 8B).

FIGS. 9A-9B are a comparison of immunogenicity to L-OMP26φNL-P6 fusion protein administered intranasally (FIG. 9A) or intraperitoneally (FIG. 9B) in the presence or absence of adjuvant. Animal model B was used.

FIGS. 10A-10B show that lipidated P6 and OMP26 and fusions L-P6ϕNL-OMP26 and LOMP26ϕNL-P6 protect against nasopharyngeal colonization (FIG. 10A) and ear infection (FIG. 10B). p-values calculated using student t-test between groups after log transforming the CFU.

FIGS. 11A-11B show L-PDϕNL-PF fusion via intranasal immunization (IN) in a modified colonization model in which no influenza infection is administered. Colonization was measured in CFU nasopharyngeally (FIG. 11A) and in ear (FIG. 11B).

FIG. 12A-12D shows effects of different lipidated proteins after intramuscular (IM) immunization as measured by nasal lavage (FIGS. 12B, 12C) and ear bullae (FIGS. 12A, 12D). FIGS. 12C and 12D are taken from the same experiment, but show LPDϕNL-OPM26 compared to control alone.

### DETAILED DESCRIPTION

Unless otherwise defined, all technical and scientific terms used herein have the same meaning as commonly understood by one of ordinary skill in the art to which this disclosure belongs. The following references provide one of skill with a general definition of many of the terms used in this disclosure: Singleton, et al., DICTIONARY OF MICROBIOLOGY AND MOLECULAR BIOLOGY (2d ed. 1994).

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH₂) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

As used herein, “vaccine” refers to a composition comprising a fusion protein as described herein, which is useful to establish immunity to Hi in the subject. It is contemplated that the vaccine comprises a pharmaceutically acceptable carrier and/or an adjuvant. In various embodiments, the vaccine comprises a vector comprising the fusion protein. It is contemplated that vaccines are prophylactic or therapeutic.

A “prophylactic” treatment is a treatment administered to a subject who does not exhibit signs of a disease or exhibits only early signs for the purpose of decreasing the risk of developing pathology. The compounds of the disclosure may be given as a prophylactic treatment to reduce the likelihood of developing a pathology or to minimize the severity of the pathology, if developed. In some embodiments, a prophylactic vaccine is administered to a subject to reduce the likelihood of developing otitis media or sinusitis in the subject.

A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective.

“Protective” immune response means that the vaccine or immunogenic composition is capable of eliciting a humoral and/or cell-mediated immune response for protecting an individual from infection. The protection provided need not be absolutely protective, i.e., the infection need not be completely prevented or eradicated, if protection is statistically significantly improved compared to a control individual population, such as an infected subject that has not been administered a vaccine or immunogenic composition. Protection can be limited to alleviating the severity or rapid onset of symptoms of the infection. In general, a “protective immune response” includes the induction of an increase in the amount of antibody specific for a particular antigen in at least 50% of individuals, including some degree of increase in the measurable functional antibody response to each antigen. In certain instances, a “protective immune response” can include a at least a 2-fold or 4-fold increase in the amount of an antibody that is specific for a particular antigen, including some degree of measurable functional antibody response to each antigen. In certain embodiments, an antibody is associated with a protective immune response. A protective immune response can be measured by assaying for the presence of antigen specific antibody in serum using assays known in the art. A protective immune response can also be assayed by measuring a percentage of bacterial count reduction in a serum bactericidal activity (SBA) assay. Such assays are also known in the art. In some embodiments, the bacterial count is reduced by at least 10%, 25%, 50%, 65%, 75%, 80%, 85%, 90%, 95% or more compared to the bacterial count in the absence of the immunogenic composition.

“Purified” as used herein refers to a fusion protein or immunogenic composition that has been isolated under conditions that reduce or eliminate the presence of unrelated materials, i.e., contaminants, including endogenous materials from which the composition is obtained. By way of example, and without limitation, a purified fusion protein is substantially free of host cell or culture components, including tissue culture or cell proteins and non-specific pathogens. In various embodiments, purified material substantially free of contaminants is at least 50% pure; at least 60%, at least 70%, at least 80%, at least 90%, at least 95%, at least 96%, at least 97%, at least 98% or even at least 99% pure. Purity can be evaluated by chromatography, gel electrophoresis, immunoassay, composition analysis, biological assay, and other methods known in the field.

As used herein, “pharmaceutical composition” refers to a composition suitable for administration to a subject animal, including humans and mammals. A pharmaceutical composition comprises a pharmacologically effective amount of a fusion protein, vaccine or immunogenic composition of the disclosure and also comprises a pharmaceutically acceptable carrier. A pharmaceutical composition encompasses a composition comprising the active ingredient(s), and the inert ingredient(s) that make up the pharmaceutically acceptable carrier, as well as any product which results, directly or indirectly, from combination, complexation or aggregation of any two or more of the ingredients. Accordingly, the pharmaceutical compositions of the present disclosure encompass any composition made by admixing a fusion protein, immunogenic composition or vaccine of the present disclosure and a pharmaceutically acceptable carrier.

As used herein, “pharmaceutically acceptable” or “pharmacologically acceptable” refers to a material which is not biologically or otherwise undesirable, i.e., the material may be administered to an individual without causing any undesirable biological effects or interacting in a deleterious manner with any of the components of the composition in which it is contained, or when administered using routes well-known in the field, as described below.

As used herein, “pharmaceutically acceptable carrier” include any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration of the active agent. Typical modes of administration include, but are not limited to, enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound or conjugate for pharmaceutical use including, e.g., metal salts (sodium, potassium, magnesium, calcium, etc.) and salts of ammonia or organic amines.

Haemophilus influenzae (Hi)

H. influenzae is a small, non-motile gram negative bacterium. Some Hi strains are polysaccharide encapsulated and capsule types are designated a, b, c, d, e and f. Nontypeable Hi) strains lack a polysaccharide capsule and are sometimes denoted as “nonencapsulated.” Noncapsulated Hi strains are genetically distinct from encapsulated strains and are more heterogeneous than the type b H. influenzae isolates. Hi presents a complex array of antigens to the human host. Possible antigens that may elicit protection include outer membrane proteins (OMPs), lipopolysaccharides, lipoproteins, adhesion proteins and noncapsular proteins. Among these antigens there are components shared by capsulated and noncapsulated strains, including proteins Omp26, P6, P4, PD and PF.

Humans are the only host for H. influenzae. Hi strains commonly reside in the upper respiratory tract including the nasopharynx and the posterior oropharynx, the lower respiratory tract and the female genital tract. Hi causes a broad spectrum of diseases in humans, including but not limited to, otitis media, pneumonia, sinusitis, septicemia, endocarditis, epiglottitis, septic arthritis, meningitis, postpartum and neonatal infections, postpartum and neonatal sepsis, acute and chronic salpingitis, epiglottis, pericarditis, cellulitis, osteomyelitis, endocarditis, cholecystitis, intraabdominal infections, urinary tract infection, mastoiditis, aortic graft infection, conjunctitivitis, Brazilian purpuric fever, occult bacteremia and exacerbation of underlying lung diseases such as chronic bronchitis, bronchietasis and cystic fibrosis.

Antigenic Hi proteins include Omp26, P6, P4, PD and PF. In some embodiments, the OMP26 protein comprises the amino acid sequence set forth in SEQ ID NO: 2. In some embodiments, the P6 protein comprises the amino acid sequence set forth in SEQ ID NO: 4. In some embodiments, the P4 protein comprises the amino acid sequence set forth in SEQ ID NO: 6. In some embodiments, the PD protein comprises the amino acid sequence set forth in SEQ ID NO: 8. In some embodiments, the PF protein comprises the amino acid sequence set forth in SEQ ID NO: 10. Fragments of any one of SEQ ID NOs: 2, 4, 6, 8 or 10 that elicit an immune response are also contemplated.

Fusion Proteins

The present disclosure provides a fusion protein comprising all or part of two or more Haemophilus influenza (Hi) proteins selected from the group consisting of OMP26, P6, P4, PD and PF, wherein at least one of the Hi proteins or fragments thereof are lipidated. Contemplated fusion proteins include, LOmp26φP6, LP4ϕOmp26, L-P6φNL-PD, L-PDφNL-PF, L-PDφNL-P6, L-P6(pNL-PD, L-OMP26φNL-PD, L-PDφNL-OMP26, and L-PFφNL-P6.

The term “lipidated” as used herein refers to a protein that comprises a lipid moiety. It is contemplated that the fusion protein is lipidated, e.g., by comprising one or more Hi proteins that comprise a lipid moiety. The term “lipidated Hi protein” as used herein refers to a Hi protein or fragment thereof that comprises a lipid moiety. An Hi protein or fragment thereof that is “non-lipidated” lacks a lipid moiety. “L” refers to lipidated, “NL” refers to non-lipidated. Exemplary lipid moieties include the fatty acids provided in Table A below.

TABLE A

| Common name | Chemical structure | C:D^{[9]} |
|---|---|---|
| Caprylic acid | CH₃(CH₂)₆COOH | 8:0 |
| Capric acid | CH₃(CH₂)₈COOH | 10:0 |
| Lauric acid | CH₃(CH₂)₁₀COOH | 12:0 |
| Myristic acid | CH₃(CH₂)₁₂COOH | 14:0 |
| Palmitic acid | CH₃(CH₂)₁₄COOH | 16:0 |
| Stearic acid | CH₃(CH₂)₁₆COOH | 18:0 |
| Arachidic acid | CH₃(CH₂)₁₈COOH | 20:0 |
| Behenic acid | CH₃(CH₂)₂₀COOH | 22:0 |
| Lignoceric acid | CH₃(CH₂)₂₂COOH | 24:0 |
| Cerotic acid | CH₃(CH₂)₂₄COOH | 26:0 |

In various embodiments, a lipidated fusion protein comprises a mixture of lipidated and non-lipidated Hi proteins. The term “lipid moiety” as used herein refers to a saturated, unsaturated or branched fatty acid with chain length of C10 to C18 (e.g., C10, C11, C12, C13, C14, C15, C16, C17 or C18). In some embodiments, the lipid moiety comprises a C18, C16, C14, C12, or C10 fatty acid. In some embodiments, the lipid moiety comprises a diacyl and/or triacyl fatty acid. In some embodiments, the lipid moiety is an N-acylated and/or O-acylated fatty acid. In various embodiments, the lipid moiety comprises a mixture of diacyl and triacyl fatty acids. In various embodiments, the mixture of diacyl and tracyl fatty acids is in a ratio of 1:1, 1.5:1, 2:1, 2.5:1, or 3:1.

In some embodiments, the fusion protein comprises Omp26 or fragment thereof and P6 or fragment thereof, wherein the P6 or fragment thereof is lipidated and the Omp26 is non-lipidated. In some embodiments, the fusion protein comprises Omp26 or fragment thereof and P6 or fragment thereof, wherein the Omp26 or fragment thereof is lipidated and the P6 is non-lipidated.

In some embodiments, the fusion protein comprises two or more Hi protein or fragments thereof that are lipidated.

In some embodiments, the fusion protein comprises all or part of Omp26 and P6, wherein the Omp26 comprises one or more C16 fatty acids. In some embodiments, the fusion protein comprises all or part of Omp26 and P6, wherein the P6 comprises one or more C16 fatty acids.

In some embodiments, the fusion protein comprises lipidated Omp26 comprising all or part of the amino acid sequence set forth in SEQ ID NO: 14. In some embodiments, the fusion protein comprises lipidated P6 comprising all or part of the amino acid sequence set forth in SEQ ID NO: 16. In some embodiments, the fusion protein comprises lipidated PD comprising all or part of the amino acid sequence set forth in SEQ ID NO: 18. In some embodiments, the fusion protein comprises lipidated PF comprising all or part of the amino acid sequence set forth in SEQ ID NO: 20.

In some embodiments, the two or more Hi proteins or fragments thereof are linked. In some embodiments, the two or more Hi proteins or fragments thereof are linked by a peptide linker. A “linker,” as used herein, refers to an amino acid or peptide sequence that is situated between two polypeptide sequences and links the two polypeptides. A linker can be from 1-80 amino acids in length. In some embodiments, a linker can be 2-40, 3-40, 3-30, or 3-20 amino acids long. In some embodiments, a linker can be a peptide of 18, 17, 16, 15, 14, 13, 12, 11, 10, 9, 8, 7, 6, 5, 4 or 3 amino acids long. In other embodiments, a linker can be 3-25, 3-18, 5-20, 6-18, or 10-20 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. In many cases, linkers lack free cysteine residues (i.e. and are therefore not involved in disulfide bonds) and also do not contain N-glycosylation sites (that is, Asn-Xxx-Ser/Thr, where X can be any amino acid except proline). Examples of other suitable linkers include G2, G3, G3S, G3P, G3q, G5, G5S, among many others. Each capital letter in the foregoing linkers refers to the conventional one-letter code for an amino acid and each number refers to the number of tandem repeats of the amino acid in the linker. For example, “G3SG2” refers to a linker having the sequence Gly-Gly-Gly-Ser-Gly-Gly. “G4S” refers to a linker having the sequence Gly-Gly-Gly-Gly-Ser. In various embodiments, the linker is a GS linker having a combination of Gly and Ser residue. In some embodiments, the linker comprises GlySerGlyGlyGlyGly (SEQ ID NO: 12).

In some embodiments, the fusion protein comprises the amino acid sequence set out in SEQ ID NOS: 29 or 31.

An immunogenic composition comprising one or more of the fusion proteins described herein is also contemplated. In some embodiments, the immunogenic composition comprises a mixture of lipidated fusion proteins. For example, in some embodiments, at least 10% of the fusion proteins in the immunogenic composition comprises a diacyl fatty acid. In some embodiments, at least 10%, 20%, 30%, 40%, 50%, 60%, 70%, 80%, 90% or more of the fusion proteins in the immunogenic composition comprise a diacyl fatty acid. In some embodiments, at least 10% of the fusion proteins in the immunogenic composition comprises a triacyl fatty acid. In some embodiments, at least 10%, 20%, 30%, 40%, 50%, 60%, 70%, 80%, 90% or more of the fusion proteins in the immunogenic composition comprise a triacyl fatty acid.

A vaccine comprising one or more of the fusion proteins described herein is also contemplated. In some embodiments, the vaccine comprises a mixture of lipidated fusion proteins. For example, in some embodiments, at least 10% of the fusion proteins in the vaccine comprises a diacyl fatty acid. In some embodiments, at least 10%, 20%, 30%, 40%, 50%, 60%, 70%, 80%, 90% or more of the fusion proteins in the vaccine comprise a diacyl fatty acid. In some embodiments, at least 10% of the fusion proteins in the vaccine comprises a triacyl fatty acid. In some embodiments, at least 10%, 20%, 30%, 40%, 50%, 60%, 70%, 80%, 90% or more of the fusion proteins in the vaccine comprise a triacyl fatty acid.

Methods of Making

The present disclosure also provides methods for making a fusion protein comprising all or part of two or more Haemophilus influenza (Hi) proteins selected from Omp26, P6, P4, PD and PF, wherein at least one of the Hi proteins comprises a lipid moiety, the method comprising (i) providing a nucleic acid sequence encoding a lipid moiety signal sequence region; (ii) providing a first nucleic acid sequence encoding all or part of Hi protein Omp26, P6, P4, PD or PF; (iii) providing a second nucleic acid sequence encoding all or part of Hi protein Omp26, P6, P4, PD or PF, wherein the second nucleic acid encodes a different Hi protein from (ii); (iv) optionally providing a third or additional nucleic acid sequences encoding all of part of one or more additional Hi proteins Omp26, P6, P4, PD or PF, wherein the third or more nucleic acids encode different Hi proteins of (ii) and (iii); (v) inserting the nucleic acid sequences (i)-(iv) into a plasmid vector capable of expressing the nucleic acids; (vi) transfecting the plasmid vector into a host cell capable of expressing the nucleic acids molecules and expressing the fusion protein; and (vii) purifying the recombinant fusion protein expressed in the host cell. In various embodiments, the host cell is grown in minimal media.

In another embodiment, contemplated herein is a method of making a fusion protein comprising all or part of two or more Haemophilus influenzae (Hi) proteins selected from the group consisting of Omp26, P6, P4, PD and PF, wherein at least one of the Hi proteins is lipidated, the method comprising: i) inserting a nucleic acid encoding a lipid moiety signal sequence region upstream of a first nucleic acid encoding all or part of a Haemophilus influenzae (Hi) protein Omp26, P6, P4, PD or PF in a plasmid vector; ii) inserting a second nucleic acid encoding all or part of a Haemophilus influenzae (Hi) protein Omp26, P6, P4, PD or PF in the plasmid vector; iii) optionally inserting a third or additional nucleic acid sequences encoding one or more additional Hi proteins or fragment thereof selected from the group consisting of Omp26, P6, P4, PD and PF; iv) transfecting the plasmid vector into a host cell capable of expressing the nucleic acid molecules; v) purifying the fusion protein expressed by the plasmid. In various embodiments, the host cell is grown in minimal media.

In various embodiments, the lipid moiety signal sequence is selected from the group consisting of MNKFVKSLLVAGSVAALAAC (SEQ ID NO: 36), with or without the terminal C residue; MQLNKVLKGLMIALPVMAIAAC (SEQ ID NO: 37), with or without the terminal C residue, MKTTLKMTALAALSAFVLAGC (SEQ ID NO: 38) or MKTTLKMTALAALSAFVLAG (SEQ ID NO: 11). In various embodiments, the lipid moiety signal sequence is a P4 signal sequence MKTTLKMTALAALSAFVLAGC (SEQ ID NO: 38) or MKTTLKMTALAALSAFVLAG (SEQ ID NO: 11).

The present disclosure also provides methods for making a fusion protein comprising all or part of two or more Haemophilus influenza (Hi) proteins selected from Omp26, P6, P4, PD and PF, wherein at least one of the Hi proteins comprises a lipid moiety, the method comprising (i) providing a nucleic acid sequence encoding a P4 lipid moiety signal sequence region; (ii) providing a first nucleic acid sequence encoding all or part of Hi protein Omp26, P6, P4, PD or PF; (iii) providing a second nucleic acid sequence encoding all or part of Hi protein Omp26, P6, P4, PD or PF, wherein the second nucleic acid encodes a different Hi protein from (ii); (iv) optionally providing a third or additional nucleic acid sequences encoding all of part of one or more additional Hi proteins Omp26, P6, P4, PD or PF, wherein the third or more nucleic acids encode different Hi proteins of (ii) and (iii); (v) inserting the nucleic acid sequences (i)-(iv) into a plasmid vector capable of expressing the nucleic acids; (vi) transfecting the plasmid vector into a host cell capable of expressing the nucleic acids molecules and expressing the fusion protein; and (vii) purifying the recombinant fusion protein expressed in the host cell. In various embodiments, the host cell is grown in minimal media.

In another embodiment, contemplated herein is a method of making a fusion protein comprising all or part of two or more Haemophilus influenzae (Hi) proteins selected from the group consisting of Omp26, P6, P4, PD and PF, wherein at least one of the Hi proteins is lipidated, the method comprising: i) inserting a nucleic acid encoding a P4 lipid moiety signal sequence region upstream of a first nucleic acid encoding all or part of a Haemophilus influenzae (Hi) protein Omp26, P6, P4, PD or PF in a plasmid vector; ii) inserting a second nucleic acid encoding all or part of a Haemophilus influenzae (Hi) protein Omp26, P6, P4, PD or PF in the plasmid vector; iii) optionally inserting a third or additional nucleic acid sequences encoding one or more additional Hi proteins or fragment thereof selected from the group consisting of Omp26, P6, P4, PD and PF; iv) transfecting the plasmid vector into a host cell capable of expressing the nucleic acid molecules; v) purifying the fusion protein expressed by the plasmid. In various embodiments, the host cell is grown in minimal media.

In some embodiments, the nucleic acid sequence encoding a Hi protein comprises a sequence that it at least 70%, 75%, 80%, 85%, 90%, 95%, 96%, 97%, 98% or 99% identical to SEQ ID NOs: 1, 3, 5, 7 or 9. In some embodiments, the nucleic acid sequence encoding Omp26 comprises SEQ ID NO: 1. In some embodiments, the nucleic acid sequence encoding P6 comprises SEQ ID NO: 3. In some embodiments, the nucleic acid sequence encoding P4 comprises SEQ ID NO: 5. In some embodiments, the nucleic acid sequence encoding PD comprises SEQ ID NO: 7. In some embodiments, the nucleic acid sequence encoding PF comprises SEQ ID NO: 9. Fragments of any one of SEQ ID NOs: 1, 3, 5, 7, or 9 that encode an Hi protein capable of eliciting an immune response are also contemplated.

In some embodiments, the nucleic acid sequence encoding a lipidated Hi protein comprises a sequence that it at least 70%, 75%, 80%, 85%, 90%, 95%, 96%, 97%, 98% or 99% identical to SEQ ID NOs: 13, 15, 17, or 19. In some embodiments, the nucleic acid sequence encoding lipidated Omp26 comprises SEQ ID NO: 13. In some embodiments, the nucleic acid sequence encoding lipidated P6 comprises SEQ ID NO: 15. In some embodiments, the nucleic acid sequence encoding lipidated PD comprises SEQ ID NO: 17. In some embodiments, the nucleic acid sequence encoding lipidated P4 comprises SEQ ID NO: 19. Also contemplated is a nucleic acid encoding any one of fusion proteins LOmp26φP6 (SEQ ID NO: 31), LP6φOmp26 (SEQ ID NO: 29), L-P6φNL-PD (SEQ ID NO: 39), L-PDφNL-PF (SEQ ID NO: 40), L-PDφNL-P6 (SEQ ID NO: 41), L-P6φNL-PD (SEQ ID NO: 42), L-OMP26φNL-PD (SEQ ID NO: 43), L-PDφNL-OMP26 (SEQ ID NO: 44), or L-PFφNL-P6 (SEQ ID NO: 45).

In some embodiments, the nucleic acid encoding the P4 lipid moiety signal sequence is inserted upstream of the first nucleic acid sequence in the plasmid vector and the second nucleic acid sequence, and optional third and additional nucleic acid sequences, are inserted downstream of the first nucleic sequence in the plasmid vector. In some embodiments, the P4 lipid moiety signal sequence comprises the nucleotide sequence set forth in SEQ ID NO: 11. In some embodiments, the P4 lipid moiety signal sequence comprises the nucleotide sequence set forth in SEQ ID NO: 38. In some embodiments, the nucleic acid encoding the P4 lipid moiety signal sequence is inserted upstream of the second nucleic acid sequence in the plasmid vector and the first nucleic acid sequence, and optional third and additional nucleic acid sequences, are inserted upstream of the second nucleic sequence in the plasmid vector. In some embodiments, the nucleic acid encoding the P4 lipid moiety signal sequence is inserted upstream of the second nucleic acid sequence in the plasmid vector and the first nucleic acid sequence, is inserted upstream of the second nucleic sequence in the plasmid vector, and the plasmid vector optionally comprises a third and additional nucleic acid sequence.

In some embodiments, the method further comprises inserting a nucleic acid sequence encoding a peptide linker between the first nucleic acid sequence and the second nucleic acid sequence an optional third and additional nucleic acid sequences. Contemplated herein are nucleic acids encoding any peptide linker described herein. In some embodiments, the linker sequence encodes GlySerGlyGlyGlyGly (SEQ ID NO: 12).

In some embodiments, the first nucleic acid sequence encodes a P6 protein or fragment thereof and the second nucleic acid sequence encodes an Omp26 protein or fragment thereof. In some embodiments, the first nucleic acid sequence encodes a Omp26 protein or fragment thereof and the second nucleic acid sequence encodes an P6 protein or fragment thereof.

In some embodiments, the nucleotide sequence encoding the fusion protein comprises SEQ ID NO: 28 or 30.

In various embodiments, the first, second, and linker nucleic acid sequence (and optionally third and subsequent nucleic acid sequences) provided herein are assembled into an expression vector, preferably under the control of a suitable promoter for expression of the mature lipoprotein, in accordance with a further aspect of the invention, which, in a suitable host organism, such as E. coli, causes initial translation of a fusion mRNA and expression of the recombinant mature lipidated fusion protein is expressed in the host organism.

Nucleic acids of the disclosure can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The expression vector can be introduced into a cell by transfection, for example.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant host cells comprising such vectors and expressing the fusion proteins described herein are also provided. The recombinant host cell may be a prokaryotic cell, for example an E. coli cell, or a eukaryotic cell, for example a mammalian cell or a yeast cell. Yeast cells include Saccharomyces cerevisiae, Schizosaccharomyces pombe, and Pichia pastoris cells. Mammalian cells include VERO, HeLa, Chinese hamster Ovary (CHO), W138, baby hamster kidney (BHK), COS-7, MDCK, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells. Recombinant protein-producing cells of the disclosure also include any insect expression cell line known, such as for example, Spodoptera frugiperda cells. In one embodiment, the cells are mammalian cells. In a certain embodiment, the mammalian cells are CHO cells.

It is contemplated that host cells are grown in media applicable for growth of the particular cell type. For example, bacteria may be cultured in LB, TB, SXYT broth or minimal media. In various embodiments, the host cells are bacteria and are grown in minimal media. In various embodiments, the minimal media is M9 media.

Protein purification methods are known in the art and utilized herein for recovery of recombinant proteins from cell culture media. For example, methods of protein purification are known in the art and can be employed with production of the fusion proteins of the present disclosure. In some embodiments, methods for protein purification include filtration, affinity column chromatography, cation exchange chromatography, anion exchange chromatography, and concentration. The filtration step may comprise ultrafiltration, and optionally ultrafiltration and diafiltration. Filtration is preferably performed at least about 5-times, more preferably 10 to 30 times, and most preferably 14 to 27 times. Affinity column chromatography, may be performed using, for example, PROSEP® Affinity Chromatography (Millipore, Billerica, Mass.). In various embodiments, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The fusion protein is preferably nanofiltered, for example, using a Pall DV 20 Nanofilter. The fusion protein may be concentrated, for example, using ultrafiltration and diafiltration. The method may further comprise a step of size exclusion chromatography.

Vaccines and Immunogenic Compositions and Routes of Administration

The present disclosure encompasses compositions comprising fusion protein(s), immunogenic compositions, or vaccines and a pharmaceutically acceptable carrier. Exemplary pharmaceutically acceptable carriers include any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration of the active agent.

The amount of fusion protein(s) or vector(s) to be administered will depend on several factors, such as route of administration, the condition of the individual, the degree of aggressiveness of the malignancy, and the particular vector employed. Also, the vector may be used in conjunction with other treatment modalities.

It is contemplated that an effective amount of the fusion protein(s), immunogenic compositions, vaccines is administered. An “effective amount” is an amount sufficient to achieve a desired biological effect such as to induce enough humoral or cellular immunity. This may be dependent upon the type of vaccine, the age, sex, health, and weight of the recipient. Examples of desired biological effects include, but are not limited to, production of no symptoms, reduction in symptoms, reduction in bacterial titer in tissues or mucosal secretions, complete protection against infection by Haemophilus influenzae, and partial protection against infection by Haemophilus influenzae.

A vaccine or immunogenic composition of the present disclosure is physiologically significant if its presence results in a detectable change in the physiology of a recipient patient that enhances at least one primary or secondary humoral and/or cellular immune response against Haemophilus influenzae. In one embodiment, a vaccine or immunogenic composition of the present disclosure is provided either before the onset of infection (so as to prevent or attenuate an anticipated infection) or after the initiation of an actual infection, and thereby protects against further bacterial infection.

Pharmaceutically acceptable carriers are well known in the field and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents or pH buffering agents. The composition can be a liquid solution, suspension, emulsion, tablet, pill, capsule, sustained release formulation, formulation for inhalation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

The precise dose of fusion protein(s), immunogenic composition, or vaccine to be employed in the formulation will also depend on the route of administration, and the nature of the patient, and should be decided according to the judgment of the practitioner and each patient's circumstances according to standard clinical techniques. The exact amount of vector or virus utilized in a given preparation is not critical provided that the minimum amount of virus necessary to produce immunologic activity is given. A dosage range of as little as about 10 mg, up to amount a milligram or more, is contemplated.

Effective doses of the fusion protein(s), immunogenic composition, or vaccine of the disclosure may also be extrapolated from dose-response curves derived from animal model test systems. In some embodiments, the immunogenic composition or vaccine described herein comprises fusion protein(s) in an amount ranging from 20-200 μg (e.g., about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, or about 200 μg).

In various embodiments the immunogenic composition further comprises an adjuvant. Exemplary adjuvants include, but are not limited to, saponin, non-ionic detergents, vegetable oil, mineral gels such as aluminum hydroxide, aluminum phosphate, surface active substances such as lysolecithin, pluronic polyols, polyanions, peptides, oil or hydrocarbon emulsions, polysaccharides, such as curdlan, chitosan, glucans, mannose, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-s-n-glycero-3-hydroxyphosphoryloxy)-ethylamine, BCG (bacille Calmette-Guerin) Corynebacterium parvum, ISCOMs, nano-beads, squalene, and block copolymers, which are contemplated for use alone or in combination.

ISCOM is an acronym for Immune Stimulating Complex, described initially in Morein et al. (Nature 308:457-460,1984). ISCOM's are a novel vaccine delivery system and are unlike conventional adjuvants. An ISCOM is formed in two ways. In some embodiments, the antigen is physically incorporated in the structure during its formulation. In other embodiments, an ISCOM-matrix (as supplied by, for example, Isconova) does not contain antigen but is mixed with the antigen of choice by the end-user prior to immunization. After mixing, the antigens are present in solution with the ISCOM-matrix but are not physically incorporated into the structure.

In one embodiment, the adjuvant is an oil in water emulsion. Oil in water emulsions are well known in the field, and have been suggested to be useful as adjuvant compositions (EP 399843; WO 95/17210, U.S. Patent Publication No. 20080014217). In one embodiment, the metabolizable oil is present in an amount of 0.5% to 20% (final concentration) of the total volume of the antigenic composition or isolated virus, at an amount of 1.0% to 10% of the total volume, or in an amount of 2.0% to 6.0% of the total volume.

In some embodiments, oil-in-water emulsion systems useful as adjuvant have a small oil droplet size. In certain embodiments, the droplet sizes will be in the range 120 to 750 nm, or from 120 to 600 nm in diameter.

In order for any oil in water composition to be suitable for human administration, the oil phase of the emulsion system comprises a metabolizable oil. The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this disclosure and can include commercially available oils such as NEOBEE® and others. A particularly suitable metabolizable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly suitable oil for use in this disclosure. Squalene is a metabolizable oil by virtue of the fact that it is an intermediate in the biosynthesis of cholesterol (Merck index, 10th Edition, entry no. 8619). Exemplary oils useful for an oil in water emulsion, include, but are not limited to, sterols, tocols, and alpha-tocopherol.

In additional embodiments, immune system stimulants are added to the immunogenic composition. Immune stimulants include: cytokines, growth factors, chemokines, supernatants from cell cultures of lymphocytes, monocytes, or cells from lymphoid organs, cell preparations and/or extracts from plants, cell preparation and, or extracts from bacteria (e.g., BCG, mycobacterium, Corynebacterium), parasites, or mitogens, and novel nucleic acids derived from other viruses, or other sources (e.g. double stranded RNA, CpG), polysaccharides, block co-polymers, nano-beads, or other compounds known in the field, used alone or in combination.

Particular examples of adjuvants and other immune stimulants include, but are not limited to, lysolecithin; glycosides (e.g., saponin and saponin derivatives such as Quil A (QS7 and QS21) or GPI-0100); cationic surfactants (e.g. DDA); quaternary hydrocarbon ammonium halogenides; pluronic polyols; polyanions and polyatomic ions; polyacrylic acids, non-ionic block polymers (e.g., Pluronic F-127); and 3D-MPL (3 de-O-acylated monophosphoryl lipid A). See e.g., U.S. Patent Publication Nos. 20080187546 and 20080014217.

In various embodiments, the adjuvant is an immune checkpoint inhibitor. In various embodiments, the immune checkpoint inhibitor is an inhibitor of CTLA-4, PD-1, PD-L1 or PD-L2. In some embodiments, the immune checkpoint inhibitor is an antibody. Antibodies to checkpoint inhibitors include ipilimumab, tremelimumab, pembrolizumab, nivolumab, atezolizumab, avelumab, and durvalumab. Antibodies specific for CTLA-4 include tremelimumab, and ipilimumab (YERVOY®), which has been approved for the treatment of melanoma. Antibodies to PD-1 include Pembrolizumab (KEYTRUDA®, Merck Sharp & Dohme Corp) and nivolumab (OPDIVO®, Bristol-Myers Squibb); and antibodies that target PD-Ll include Atezolizumab (TECENTRIQ®), Avelumab (BAVENCIO®), and Durvalumab (IMFINZI®).

Many methods may be used to administer or introduce the fusion protein(s), immunogenic compositions, vaccines into individuals (i.e., including subjects or patients), including but not limited to, oral, intradermal, intramuscular, intraperitoneal, intravenous, subcutaneous, and intranasal routes.

Methods of Use

The present disclosure provides a method of treating or preventing disease associated with a nontypeable Haemophilus influenza (Hi) infection comprising administering a vaccine or immunogenic composition comprising a fusion protein described herein. Diseases associated with Hi infection include, but are not limited to, otitis media, bronchitis, pneumonia, sinusitis, septicemia, endocarditis, epiglottitis, septic arthritis, meningitis, postpartum and neonatal infections, postpartum and neonatal sepsis, acute and chronic salpingitis, epiglottis, pericarditis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic bronchitis, and exacerbation of underlying lung diseases such as chronic bronchitis (AECB), bronchiectasis, and cystic fibrosis, chronic obstructive pulmonary disorder (COPD), and acute exacerbations of COPD (AECOPD). In some embodiments, the disease associated with a non-typeable Hi infection is otitis media. In some embodiments, the disease associated with a non-typeable Hi infection is acute exacerbation of chronic bronchitis (AECB). In some embodiments, the disease associated with a non-typeable Hi infection is acute exacerbation of chronic obstructive pulmonary disorder (AECOPD).

In some embodiments, the immunogenic composition is administered concomitantly with other vaccines, for example with parenteral vaccines such as DTPw or DTPa vaccines (vaccines against Bordetella pertussis—whooping cough, diphtheria, tetanus), vaccines against Haemophilus influenza b-induced meningitis, hepatitis B, or measles, mumps, rubella (MMR), vaccines against Streptococcus pneumoniae, in order to optimize the number of visits to the doctor.

Also provided is a composition comprising a fusion protein, vaccine or immunogenic composition described herein for use in treating or preventing disease associated with a nontypeable Haemophilus influenza (Hi) infection. In various embodiments, the disclosure provides for use of a composition comprising a fusion protein, vaccine or immunogenic composition described herein in the preparation of a medicament for use in treating or preventing disease associated with a nontypeable Haemophilus influenza (Hi) infection.

In various embodiments, the vaccine or immunogenic composition reduces or prevents colonization in one or more of the sinus, lungs and ears.

Packaging and Dosage Form

The fusion proteins, immunogenic compositions, or vaccines described herein may be packaged in unit dose or in multiple doses (e.g., 2 doses, 4 doses, or more than 4 doses). For multiple dose formats, the vial is typically, but not necessarily, superior to a pre-filled syringe. Suitable multiple dose forms include, but are not limited to, 2 to 10 doses per container, 0.1 to 2 mL per dose. In certain embodiments, the dose is a 0.5 mL dose.

The composition may be provided in a vial or other suitable storage container, or may be provided in a pre-fill transfer device, such as a single or multi-component syringe, which may or may not be provided with the needle. Syringes typically, but not necessarily, contain a single dose of the preservative-containing immunogenic composition of the present invention, but multiple dose pre-filled syringes are also contemplated. Likewise, the vial can include a single dose or can include multiple doses.

The effective dose volume can be established routinely, but a typical dose of the injectable composition has a volume of 0.5 mL. In certain embodiments, the dose is formulated for administration to a human subject. In certain embodiments, the dosage is formulated for administration to a human, adult, adolescent, infant, or toddler (i.e., no more than one year old) human subject and in a preferred embodiment may be administered by injection.

The liquid immunogenic compositions disclosed herein are also suitable for reconstituting other immunogenic compositions provided in lyophilized form. When the immunogenic composition is intended to be reconstituted for use as such, the present invention provides a kit having two or more vials, two or more spare fill syringes, or one or more vials and an alternate fill syringe. Group, wherein the contents of the syringe are used to restore the contents of the vial prior to injection, or vice versa.

Alternatively, the immunogenic compositions disclosed herein may be lyophilized and reconstituted, for example, using one of the many methods well known in the art for freeze drying to form dry, regular (e.g., spherical) particles, such as microparticles or Microspheres having particle characteristics, such as mean diameter dimensions, that can be selected and controlled by varying the precise method used to prepare the particles. The immunogenic composition may additionally comprise an adjuvant which may be prepared with or contained in each of the dry, regular (e.g., spherical) particles, such as microparticles or microspheres, as appropriate. In these embodiments, the disclosure further provides an immunogenic composition kit comprising a first component comprising a stable dry immunogenic composition, optionally comprising one or more preservatives, as the case may be; A second component for reconstituting the sterile aqueous solution of the first component. In certain embodiments, the aqueous solution comprises one or more preservatives, and optionally at least one adjuvant (see, for example, WO 2009/109550 (incorporated herein by reference)).

In another embodiment, the multiple dose form of the container is selected from one or more of the group consisting of, but not limited to, general laboratory glassware, flasks, beakers, graduated cylinders, decanters, bioreactors, tubing, pipe, bag, bottle, vial, vial closure (e.g., rubber stopper, screw cap), ampoule, syringe, double or multi-chamber syringe, syringe stopper, syringe plunger, rubber fasteners, plastic fasteners, glass fasteners, cartridges and disposable pens and the like. The container is not limited by the materials of manufacture and includes various materials such as glass, metal (e.g., steel, stainless steel, aluminum, etc.) and polymers (e.g., thermoplastics, elastomers, thermoplastic elastomers). In a particular embodiment, the container of this form is a 5 mL Schott 1 type glass vial with a butyl stopper. Those skilled in the art will appreciate that the above-described forms are in no way an exhaustive list, but merely serve as a guide to the skilled artisan with regard to the many forms that can be used in the present invention. Other forms contemplated for use in the present invention can be found in laboratory equipment suppliers and manufacturers, such as the United States Plastic Corp. (Lima, OH), VWR publication catalog.

Additional aspects and details of the disclosure will be apparent from the following examples, which are intended to be illustrative rather than limiting.

### EXAMPLES

### Example 1

### Cloning of Recombinant HirV Lipoproteins

All of the Haemophilus influenza recombinant vaccine (HirV) candidates in their native Haemophilus influenzae host contain a signal sequence that directs the protein to the membrane but only P6, PD and P4 are natural lipoproteins. The constructs described below have their natural signal sequences replaced with the P4 lipid-encoding signal sequence (SEQ ID NO. 11). The initial transformation of the clones was into DH5 E. coli cells. Restriction analysis and DNA sequencing of the recombinant isolated plasmid DNA was performed to confirm the cloning.

Lipidated Omp26 (LOmp26): The mature Omp26 gene containing the P4 lipid encoding N-terminal signal sequence (SEQ ID NO: 13) was originally synthesized and cloned into the Kpn1 & Sph1 sites in pBAD18Cm at GenScript (Piscataway, NJ). Primers were designed (see Table 1) and used to PCR the Omp26 gene from pBAD18Cm and clone into the Nde1 and Xho1 sites in pET21a vector that added a poly His-tag at the carboxy end of the protein. The full-length LOmp26 amino acid sequence is provided in SEQ ID NO: 14.

Lipidated P6 (LP6): The mature P6 gene sequence containing the P4 lipid encoding N-terminal signal sequence (SEQ ID NO: 15) was originally synthesized and cloned into the Kpn1 & Sph1 sites in pBAD18Cm at GenScript (Piscataway, NJ). Primers were designed (see Table 1) and used to PCR the P6 gene from pBAD18Cm and clone into the Nde1 and Xho1 sites in pET21a vector that added a poly His-tag at the carboxy end of the protein. The full-length LP6 amino acid sequence is provided in SEQ ID NO: 16.

Lipidated PD (LPD): The mature PD gene sequence containing the P4 lipid encoding N-terminal signal sequence (SEQ ID NO: 17) was originally synthesized and cloned into the Kpn1 & Sph1 sites in pBAD18Cm at GenScript (Piscataway, NJ). Primers were designed (see Table 1) and used to PCR the PD gene from pBAD18Cm and clone into the Nde1 and Xho1 sites in pET21a vector that added a poly His-tag at the carboxy end of the protein. The full-length LPD amino acid sequence is provided in SEQ ID NO: 18.

Lipidated P4 (LP4): The matureP4 gene sequence containing the P4 lipid encoding N-terminal signal sequence (SEQ ID NO: 19) was synthesized and cloned directly into the Nde1 and Xho1 sites in pET-21a(+) at GenScript (Piscataway, NJ). The P4 gene also contained a N218Q mutation to reduce its phophomonoesterase activity as previously reported. The full-length LP4 amino acid sequence is provided in SEQ ID NO: 20.

TABLE 1
PCR Primers

| | | |
|---|---|---|
| Nde1-LP4 | gatatacatATGAAGACCACCC | SEQ ID NO: 21 |
| Fwd | TGAAAATG | |
| Xho1 Omp26 | ctatatctcgagTTTCTTTTCT | SEQ ID NO: 22 |
| Rev | TGCACT | |
| Nde1 Omp26 | gatatacatATGGAGGAAAAGA | SEQ ID NO: 23 |
| Fwd | TCGCGTTC | |
| Xho1 P6 Rev | ctatatctcgagATACGCCAGA | SEQ ID NO: 24 |
| | ACCGCA | |
| Nde1 P6 Fwd | gatatacatATGAGCAGCAGCA | SEQ ID NO: 25 |
| | ACAACGATG | |
| Xho1 PD Rev | ctatatctcgagTTTGATACCC | SEQ ID NO: 26 |
| | TTCAGAA | |
| Nde1 PD Fwd | gatatacatATGAGCAGCCACA | SEQ ID NO: 27 |
| | GCAGCAAC | |

### Example 2

### Expression and Purification of Recombinant Proteins

The recombinant clones produced as described in Example 1 were transformed into BL21 or BLR expression cell lines that are B/r E. coli and lack the cytoplasmic lon protease. The lipoprotein clones were then transformed into C41(DE3) or C43(DE3) that overcome toxicity of over-produced membrane proteins compared to BL21(DE3) (Mirouz et al., J. Mol. Biol., 260:289-298, 1996); Dumon-Seignovert et al., Protein Exp. Purif., 37:203-206, 2004; Chen et al., Vaccine, 27:1400-1409, 2009). Table 2 lists the genotypes of various E. coli strains.

TABLE 2

| E. coli strain | Genotype |
|---|---|
| BL21(DE3) | F⁻ ompT hsdS_{B}(r_{B}⁻m_{B}⁻) gal dcm (DE3) |
| BL21 Star(DE3) | F⁻ ompT hsdS_{B}(r_{B}⁻m_{B}⁻) gal dcm rne131 (DE3) |
| BL21(DE3)pLysS | F⁻ ompT hsdS_{B}(r_{B}⁻m_{B}⁻) gal dcm (DE3) pLysS |
| | (Cam^{R}) |
| BLR(DE3) | F⁻ ompT hsdS_{B}(r_{B}⁻m_{B}⁻) gal lac dcm (DE3) Δ(srl- |
| | recA)306::Tn10 (Tet^{R}) |
| BLR(DE3)pLysS | F⁻ ompT hsdS_{B}(r_{B}⁻m_{B}⁻) gal lac dem (DE3) Δ(srl- |
| | recA)306::Tn10 pLysS (Cam^{R}, Tet^{R}) |
| C41(DE3) | BL21(DE3) unknown mutation |
| C43(DE3) | C41(DE3) additional unknown mutation |

(DE3): T7 polymerase (I gene) inserted in the lambda phage integrase gene. Expression of T7 polymerase is under lacUV5 control (IPTG inducible) produced from the λ-lysogen.

Cells grown in Luria-Bertanni (LB) media+100 μg/ml ampicillin in 50 ml culture tube at 37° C. shaking at 200 rpm. A 1:100 dilution of overnight grown bacteria to fresh LB media+100 ug/ml ampicillin where grown to OD₆₀₀˜0.5 (˜2.5-3 hrs at 37° C., 200 rpm) and then induced with 0.4 mM IPTG. Cells were grown for 4 hours at 30° C. and harvested by centrifugation at 10K rpm. The pellets were stored at −20° C. All of the recombinant proteins were expressed using the above conditions except for the following modifications:
LP6: C43 (DE3) cells were used.
LPD: C41(DE3)pLysS cells were used. Also, pre-growth, growth and incubation media also contained 34 μg/mL chloramphenicol.
LPF: C41(DE3)pLysS cells were used. Also, pre-growth, growth and incubation media also contained 34 μg/mL chloramphenicol. Growth and induction media also contained 1% glucose. Induction was for 4 hours at 37° C.
LP4: C43 (DE3) cells were used. Growth and induction media also contained 1% glucose.

Cell Lysis: For each thawed protein pellet produced from a 1 L batch, added 30 ml of buffer containing 50 mM Tris/300 mM NaCl/100 μg/ml lysosome pH=8. Protease inhibitor PMSF was added to 1 mM concentration. After suspending the pellet, cells were lysed with sonication for 15 sec×3-4 cycles on 30 duty cycle on ice (W-225 Sonicator Ultrasonic W-225) and incubated in waterbath @37° C., 30 min. Spin 10K rpm for 1 hr.

Next, for each thawed protein pellet produced from a 1 L batch, added 30 ml of buffer containing 50 mM Tris/300 mM NaCl/100 μg/ml lysosome pH=8. Protease inhibitor PMSF was added to 1 mM concentration. After suspending the pellet, cells were lysed with sonication for 15 sec×3-4 cycles on 30 duty cycle on ice (W-225 Sonicator Ultrasonic W-225) and incubated in waterbath @ 37° C., 30 min. Spin 10K rpm for 1 hr. For each thawed protein pellet produced from a 1 L batch, added 30 ml of buffer containing 50 mM Tris/300 mM NaCl/100 μg/ml lysosome pH=8. Protease inhibitor PMSF was added to 1 mM concentration. After suspending the pellet, cells were lysed with sonication for 15 sec×3-4 cycles on 30 duty cycle on ice (W-225 Sonicator Ultrasonic W-225) and incubate in waterbath at 37° C., 30 min. Spin 10K rpm for 1 hr.

Purification of lipidated recombinant proteins: For each thawed protein pellet produced from a 1 L batch, 30 ml of buffer containing 50 mM Tris/300 mM NaCl/100 μg/ml lysosome pH=8, was added. Protease inhibitor PMSF was added to 1 mM concentration. After suspending the pellet, cells were lysed with sonication for 15 sec×3-4 cycles on 30 duty cycle on ice (W-225 Sonicator Ultrasonic W-225), incubated in waterbath at 37° C., 30 minutes and spun at 10K rpm for 1 hour.

For binding and elution of His-tagged lipoproteins, lysed protein extract was added to equilibrated beads and collect flow through for further analysis by SDS-PAGE gel. Beads were washed with 5× column volume of wash buffer. Next, lipoproteins were eluted with 1 ml -5 ml of elution buffer (250 mM immidizole +0.5% zwittergent) and collect 1 ml fractions. Fractions were run on Nanodrop to determine protein concentration.

Fractions were analyzed by SDS-PAGE for recovery and purity of lipoprotein. If needed, size exclusion or ion-exchange were performed to further purify the lipoprotein.

Millipore Centricon tube filters (3 or 10 kDa) were used to remove immidazole. Basically, the lipoprotein fraction was added to the filter and spun at 3K rpm for 20-30 min. 10-15 mls of PBS buffer containing 0.05% zwittergent was added to 1 ml of concentrate and spun again. This was repeated 2 times.

The protein concentration was determined using Bradford assay and diluted with PBD buffer containing 0.05% zwittergent to a final concentration of >0.5 mg/ml and stored aliquots at −80° C. Size and purity of the lipoproteins was assessed by SDS-PAGE (data not shown). The final yield of the lipoproteins was 1-4 mg per liter of induced culture.

Purification of non-lipidated recombinant proteins: All of the non-lipidated proteins were expressed using the same condition as the lipoproteins. During lysis, no lysozyme was added. However, 1% Triton was added before sonication. After the first centrifugation, the supernatant containing the proteins was purified using the Ni-beads protocol as above but without added zwittergent in the buffers.

Final yield of non-lipidated proteins was 5-15mg per liter of inducted culture.

### Example 3

### Mass Spectrometry Analysis

Lipidated proteins were prepared for LC-MS analysis by reduction and alkylation followed by protein precipitation. After precipitation, proteins were digested to peptides using trypsin and chymotrypsin separately and a small sample volume run on a nanoLC system using two different gradients to ensure that the highly lipidated peptides were eluted completely. This was followed by mass spectra generation using HCD-IT (Ion Trap) and HCD-OT (Orbitrap). Data processing is done using Proteome Discoverer 2.2. A combination of digestion using trypsin, high organic solvent gradient, and MS using HCD-IT (Ion Trap) shows the best results in identifying lipidated peptides.

A control triacylated peptide, PAM3CSK4, was run to optimize detection of triacylated peptides generated from the recombinant lipidated proteins. Results showed high confidence detection of the fully triacylated peptide. Also detected were O-linked diacylated and N-linked monoacylated products that generated during collision/fragmentation process.

Theoretical monoisotopic and average masses for each of the predicted lipid compositions (1,2,3,A,A′,B,B′,C,D,E), based on published literature, was used by Skyline and by Proteome Discoverer software in analysis of the generated spectra. Modifications included neutral mass (M), +1 charge (M+H), +2 charge (M+2 detection of O-linked diacylated (Diacylglycerol) with many PSMs for lipid structures 1, 2, 3 and B. Lipid structure D also generated high confidence with only one PSM due to its peak being clearly defined (data not shown). Triacylated rLP6 was detected with medium confidence with 1 PSM and for only lipid D structure. Table 3 shows a summary MS analysis of 2 different lots of LP6 and LP4, and 1 lot of LPF.

TABLE 3
Summary of MS samples analyzed

| | Triacyl (Diacylglycerol + | | | | | | Monoacyl (N-linked | |
|---|---|---|---|---|---|---|---|---|
| | palmitoyl) | | | Diacyl (O-linked diacylglycerol) | | | palmitoyl) | |
| Peptide/Protein | Type | Confidence | PSMs | Type | Confidence | PSMs | Confidence | PSMs |
| PAM3CSK4 | | High | 247 | | High | 132 | High | 259 |
| LP6, #1 | D | Medium | 2 | 1 | High | 33 | Medium | 3 |
| | | | | 2 | High | 26 | | |
| | | | | 3 | High | 39 | | |
| | | | | B | High | 14 | | |
| | | | | D | High | 1 | | |
| LP6, #2 | D | Medium | 1 | 1 | High | 10 | — | — |
| | | | | 2 | High | 5 | | |
| | | | | B | High | 3 | | |
| | | | | D | High | 1 | | |
| LP4, #1 | — | — | — | 2 | Medium | 3 | — | — |
| | | | | 3 | Medium | 7 | | |
| LP4, #2 | 3 | Medium | 5 | 2 | Medium | 3 | — | — |
| LPF | 3 | Medium | 1 | 1 | Medium | 5 | — | — |
| | | | | 2 | Medium | 4 | | |
| | | | | 3 | Medium | 4 | | |

### Example 4

### The Addition of N-Acetyltransferase Enzyme Enhances Triacylation

Lipoproteins produced in Gram-negative and certain Gram-positive bacteria possess both O-acylated and N-acylated fatty acids.^{1,2} Addition of the N-acylated fatty acid occurs after diacylation of glycerol at the N-terminal cysteine residue.³ In E. coli, the enzyme, N-acyltransferase (Lnt, originally named CutE)⁴ transfers the sn-1 -acyl chain of phosphatidylethanolamine to the amine group of cysteine generating a triacylated lipoprotein. The crystal structure of Lnt has been determined and is a monomeric 8-transmembrane protein localized to the inner-membrane.⁵

The level of recombinantly expressed triacylated versus diacylated lipoprotein produced in E. coli may reflect the amount of Lnt available to catalyze the final step in maturation of the lipoprotein.

The approach is to clone the Lnt gene with it natural Shine Delgarno, ribosome-binding region, sequence from E. coli BLR21 cells into a compatible plasmid to our pET21 expression vectors. A p15A plasmid origin vector such as pACYC184, which is a low copy plasmid, and to use a noninducible promoter will be used in case overexpression of Lnt is lethal. The chloramphenicol resistance gene on pACYC184 will be replaced with the lnt gene expression of Lnt will be under constitutive control of the chloramphenicol promoter. The pACYC184_Lnt plasmid will be cloned in DH5 competent cells and tested for expression of the plasmid-encoded lnt RNA. After confirmation of the correct clone and RNA expression, the plasmid will then be transformed into the HirV recombinant lipoprotein ampicillin resistant C41(DE3) or C43(DE3) strains and co-selected for tetracycline resistance. The level of triacylation of our HirV purified lipidated proteins will be determined by mass spectrometry (MS), such as by the methods described in Example 2.

References for Example 4:
1. Nguyen M T, Gotz F. Lipoproteins of Gram-Positive Bacteria: Key Players in the Immune Response and Virulence. Microbiol Mol Biol Rev 2016; 80(3): 891-903.
2. Nakayama H, Kurokawa K, Lee B L. Lipoproteins in bacteria: structures and biosynthetic pathways. The FEBS journal 2012; 279(23): 4247-68.
3. Okuda S, Tokuda H. Lipoprotein sorting in bacteria. Annu Rev Microbiol 2011; 65: 239-59.
4. Gupta S D, Gan K, Schmid M B, Wu HC. Characterization of a temperature-sensitive mutant of Salmonella typhimurium defective in apolipoprotein N-acyltransferase. J Biol Chem 1993; 268(22): 16551-6.
5. Noland C L, Kattke M D, Diao J, et al. Structural insights into lipoprotein N-acylation by Escherichia coli apolipoprotein N-acyltransferase. Proc Natl Acad Sci U S A 2017; 114(30): E6044-e53.
6. Luo Y, Friese O V, Runnels H A, et al. The Dual Role of Lipids of the Lipoproteins in Trumenba, a Self-Adjuvanting Vaccine Against Meningococcal Meningitis B Disease. The AAPS journal 2016; 18(6): 1562-75.
7. Martinon-Torres F, Gimenez-Sanchez F, Bernaola-Iturbe E, Diez-Domingo J, Jiang Q, Perez J L. A randomized, phase ½ trial of the safety, tolerability, and immunogenicity of bivalent rLP2086 meningococcal B vaccine in healthy infants. Vaccine 2014; 32(40): 5206-11.

### Example 5

### Cloning of Recombinant Lipidated Fusion Proteins

The following Example describes the process for generation LP6ϕOmp26 and LOmp6ϕP6 fusion proteins.

LP4ϕOmp26 fusion protein: The P6 and Omp26 genes used for making the fusion were from pET21 clone constructs described in Example 1. The fusion linker sequence is provided in SEQ ID NO: 12. To make the LP6ϕOmp26 construct, Nde1-LP4 Fwd & Lnk-P6 Rev primers were used to PCR the LP6 DNA fragment (see Table 1 for primer sequences). In a separate reaction, Lnk-Omp26 Fwd & Xho1 Omp26 Rev primers were used to PCR the Omp26 DNA fragment. The purified DNA fragments were added together and after an initial PCR reaction, primers Nde1-LP4 Fwd & Xho1Omp26 Rev were added to the PCR reaction. The purified final fused PCR product was digested with Nde1 & Xho1 and cloned into pET21 at the Nde1 & Xho1 sites. The nucleotide cloned into pET21a is set forth in SEQ ID NO: 28 and the encoded amino acid sequence is set forth in SEQ ID NO: 29.

LOmp26ϕP6 fusion protein: The LOmp26 and P6 genes used for making the fusion were from pET21 clone constructs described in Example 1. The fusion linker sequence is provided in SEQ ID NO: 12. To make the LOmp260P6 construct, Ndeϕ-LP4 Fwd & Lnk-Omp26 Rev primers were used to PCR the LOmp26 DNA fragment (see Table 1 for primer sequences). In a separate reaction, Lnk-P6 Fwd & Xho1 P6 Rev primers were used to PCR the P6 DNA fragment. The purified DNA fragments were added together and after an initial PCR reaction, primers Nde1 -LP4 Fwd & Xho1 P6 Rev were added to the PCR reaction. The purified final fused PCR product was digested with Nde1 & Xho1 and cloned into pET21 at the Nde1 & Xho1 sites. The DNA sequence cloned into pET21a is set forth in SEQ ID NO: 30 and the predicted full-length His-tag fusion protein sequence is set forth in SEQ ID NO: 31.

### Example 6

### Cloning of Recombinant Non-Lipidated Fusion Proteins

The following Example describes the process for generation non-lipidated P6ϕOmp26 and Omp6ϕP6 fusion proteins.

P6ϕOmp26 : The P6 and Omp26 genes used for making the fusion were from pET21 clone constructs described in Example 1. The fusion linker sequence is set forth in SEQ ID NO: 12. To make the P6ϕOmp26 construct, Nde1 P6 Fwd & Lnk-P6 Rev primers were used to PCR the P6 DNA fragment (see Table 1 for primer sequences). In a separate reaction, Lnk-Omp26 Fwd & Xho1 Omp26 Rev primers were used to PCR the Omp26 DNA fragment. The purified DNA fragments were added together and after an initial PCR reaction, primers Nde1 P6 Fwd & Xho1 Omp26 Rev were added to the PCR reaction. The purified final fused PCR product was digested with Nde1 & Xho1 and cloned into pET21 at the Nde1 & Xho1 sites. The DNA sequence cloned into pET21a is set forth in SEQ ID NO: 32 and the predicted full-length His-tag fusion protein sequence is set forth in SEQ ID NO: 33.

Omp6ϕP6—The Omp26 and P6 genes used for making the fusion were from pET21 clone constructs described in this report. The fusion linker sequence is set forth in SEQ ID NO: 12. To make the Omp260ϕP6 construct, Nde1 Omp26 Fwd & Lnk-Omp26 Rev primers were used to PCR the Omp26 DNA fragment (see Table 1 for primer sequences). In a separate reaction, Lnk-P6 Fwd & Xho1 P6 Rev primers were used to PCR the P6 DNA fragment. The purified DNA fragments were added together and after an initial PCR reaction, primers Nde1 Omp26 Fwd & Xho1 P6 Rev were added to the PCR reaction. The purified final fused PCR product was digested with Nde1 & Xho1 and cloned into pET21 at the Nde1 & Xho1 sites. The DNA sequence cloned into pET21a is set forth in SEQ ID NO: 34 and the predicted full-length His-tag fusion protein sequence is set forth in SEQ ID NO: 35.

### Example 7

### Immunogenicity Study of Lipidated vs. Non-Lipidated Forms of P6 and Omp26

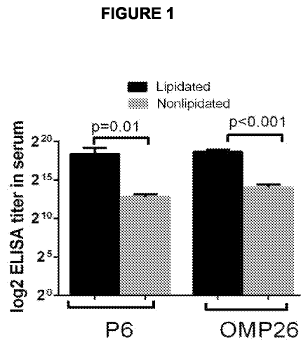

Mice (n=4-5) were vaccinated at designated times with 10 μg of dose for each proteins and sera was collected 2 weeks after the 3rd doses. Results showed that significantly higher mouse serum-IgG antibodies were induced by lipidated compared to nonlipidated versions of P6 and OMP26 (FIG. 1).

### Example 8

### Immunogenicity Study of Lipidated Fusion Proteins

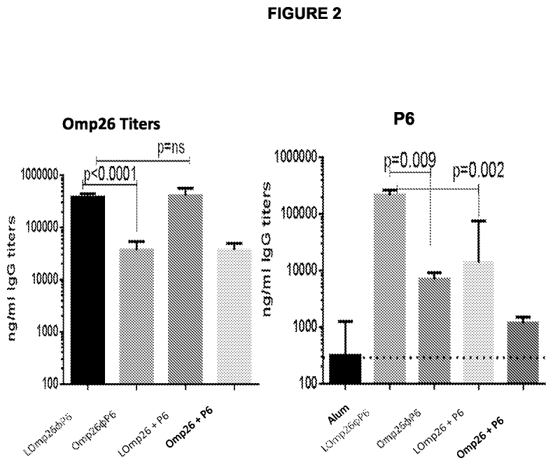

Fusion constructs of OMP26ϕP6 protein where the signal sequence was attached to OMP26 (LOmp26ϕP6) were generated as described in Example 5. Mice (n=4-5) were vaccinated at designated times with 10 μg of dose of LOmp26ϕP6 or non-lipidated Omp26ϕP6 and sera was collected 2 weeks after the 3rd doses. Results indicate that LOmp26ϕP6 increased IgG response to both OMP26 and P6 even though P6 was not lipidated (FIG. 2). IgG OMP26 titers indicate that LOMP26 is more immunogenic and showed no difference in immunogenicity when it is fused to other proteins, indicating the fusion construct still maintains its original fold and structure. Interestingly significant increases in P6 IgG titers were observed when it is fused to LOMP26 compared to the nonfused proteins demonstrating trans effect and similar functionality of the fused construct.

### Example 9

### Otitis Media Animal Model

A mouse Hi acute otitis media (AOM) model mimicking natural human infection pathogenesis was made by introducing an NP viral infection one week prior to Hi challenges using mouse adapted flu strain PR8/36 (PR8). Data shows that Hi NP colonization density exceeds the pathogenic threshold to establish AOM and we have validated this model in vaccine protection using Protein D.

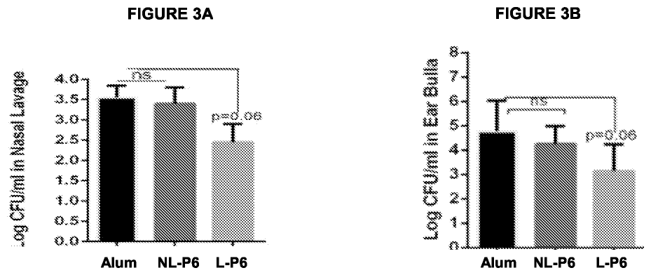

Using heat-killed Hi as an immunogen (positive control), results showed a very significant reduction in NP colonization and complete protection against AOM. In challenge experiments in this mouse model (n=4-5 mice per group) with lipidated and nonlipidated P6 proteins, lipidated P6 resulted in >1 log reduction of NP colonization of Hi (FIG. 3). Hi burden was lowest in mice receiving lipidated P6 in both the nasal lavage and ear bulla samples.

### Example 10

### Additional In Vivo Studies

Lipidated and non-lipidated proteins of P6, OMP26, OMP26ϕP6 and P6ϕOMP26 fusion are expressed in E. coli strains BLR21(DE3) and C43(DE3) respectively and purified using standard established protocols (Fletcher et al., Infection and Immunity, 74:6383-6845, 2005). For purification of lipidated proteins, 1% zwittergent are used during cell lysis to extract proteins from membrane and the final lipidated construct protein stored at −80° C. in zwittergent buffer to keep the protein stable. Mass spectrometry analysis is performed on all 4 lipidated proteins to confirm is triacylation status (complete palmitoylation).

C57BL/6 mice (n=10, 5 male and 5 female/group) at 4 weeks of age are immunized intramuscularly with two different concentrations of vaccine formulation containing 10 ug and 25 μg of each protein (molar amount of each protein including fusion construct will be considered in calculation) (Vertebrate section) with aluminum phosphate adjuvant on day 0, 7 and 21. Control mice receive A1PO4 only (negative control) or HK-Hi (positive control). In one group, both P6 and OMP26 proteins are mixed in lipidated and non-lipidated versions (for example L-OMP26+NL-P6, L-P6+NL-OMP26 and NLP6+NLOMP26) and are tested for synergistic affect in challenge compared to fusion construct. Blood samples taken on day 35 and serum IgG and IgM are measured by standardized-ELISA against the individual nonlipidated P6 and OMP26 proteins. At day 35, the mice are infected with PR8 virus using the dose in the above standard model of Hi AOM33 and one week later, the mice will be challenged intranasally with 10 μl of Hi strain 575 given to each nostril (Strain 575 is from repository that has been characterized for surface expression of both proteins and whole genome sequenced). Three days after infection, ear lavage, ear bullae, nasal wash (NW), NP mucosa tissue (embedded in paraffin for preservation), NALT, blood and spleen are taken. Measurement of Hi bacteria in NW and middle ear samples are enumerated by plating on chocolate plates with different dilutions.

Next, mucosal IgG and IgA antibody levels are determined from the nasal wash (NW) and ear lavage fluids. Nasal associated lymphoid tissue (NALT) is isolated, which is the principal mucosal site of respiratory tract infections and reservoir for local T-cells. Spleens are homogenized and isolated cells stored in liquid nitrogen for T cell analysis.

From the nasal and ear lavage, levels of IL-17, IL-6 and IL-22, which are produced from memory Th17 cells, are determined using Luminex (Biorad). In humans, IL-6 is not produced from Th17 cells. For measuring Th17 cell response, CD4+ T-cells will be isolated from spleen (using MACS microbead technology; miltenyibiotec) and Th17 cells quantitated by intra cellular staining (ICS) of IL-17A. Duplicate assays is not performed, as numbers of mice tested in each group are their own control for variance. Anti-pathogenic Th17 response in the NALT (by pooling NALT from 2-3 mice from each group to get sufficient # of cells) against lipidated and non-lipidated proteins is also determined by standard ICS staining.

Without wishing to be bound to any particular theory, it is contemplated that reduced Hi in the ears and NP samples from mice vaccinated with lipidated proteins as described herein (alone or as fusion constructs) compared to nonlipidated antigens will be observed.

Next, from the stored (-20° C.) nasal and middle ear lavage of mice vaccinated as described above, IL-8 and TNF-α levels, known chemokines for neutrophils, are determined using Luminex (Biorad). Neutrophil infiltrate is determined by histopathology of NP mucosal tissue as described previously (Lu et al., PLoS Pathogens, 4:e1000159, 2008; van Rossum et al., Infect. Immun., 73:718-7726, 2005). To confirm that neutrophils are a major player responsible for reduction of Hi in the NP, immunized neutrophil depleted mice are assayed for diminished protection. Six mice per group are immunized with lipidated and nonlipidated P6 and OMP26 and treated with monoclonal antibody, RB6-8C5, at time of challenge as described previously for Spn carriage (Lu, supra) and observed for protection difference against Hi in NP and middle ear. Antibody, cytokine levels and neutrophil number are correlated with bacterial burden of Hi in NP and ME lavage.

Without wishing to be bound to any particular theory, it is contemplated that higher IL-8 and TNF-α levels and neutrophil infiltrate in the nasal wash and mucosa will be observed after challenge from mice immunized with lipidated vaccine antigens. Depletion of neutrophils at time of challenge will reduce the protection. Similar results compared with lipidated and non-lipidated protein vaccinations are expected.

### Example 11

### Passive Immunization

The following Example provides a protocol to determine whether passive transfer of immune sera alone and/or immune T-cells confer protection in naïve infant and adult mice. 10-adult mice/group are vaccinated (following schedule described in Example 9) with lipidated and non-lipidated P6 and OMP26 proteins. Two weeks after the 3rd dose of vaccination, mice are sacrificed and blood and spleen collected and processed for serum and spleen cells. Antibody levels are quantitated and serum samples with high titers for each vaccinated proteins are pooled. Sera samples containing 10 μg of antibodies are adoptively transferred to naïve mice via tail vein. A second group of naïve mice receive isolated immune CD4+ T-cells alone, and a third group of naive mice receive combined immune serum and CD4+ T-cells, and are given to infant (day 14) and adult (6 week) mice via tail vein. Seven days before transfer, mice (n=8 in each group) receive PR8//36 and 7 days later challenged with Hi 575 strain (4-6 hours after antibody and T-cell transfer). Bacterial loads in the ears and nasal lavage are determined on day 3 post challenge.

Without wishing to be bound to any particular theory, it is contemplated that protection in adult mice will only require CD4+ T-cells. Adopted transfer of antibody and CD4+ T-cells will better passively protect infant mice from colonization and AOM.

### Example 12

### Effects of Tri- or Di-acylation on Immune Response

Methods of producing proteins can result in varied levels of protein lipidation. To determine if the type of lipidation had any effect on the immunogenicity of the lipidated Hi proteins, the types and estimated levels of lipidation on the fusion proteins was examined.

To look at the effects of lipidation, HEK-BLUE™ hTLR2-TLR1 cells (SEAP reporter 293 cells expressing the human TLR2 and TLR1 gene, (InvivoGen, San Diego, CA), which respond to high levels of triacylated peptides, were cultured according to manufacturer's recommended protocol.

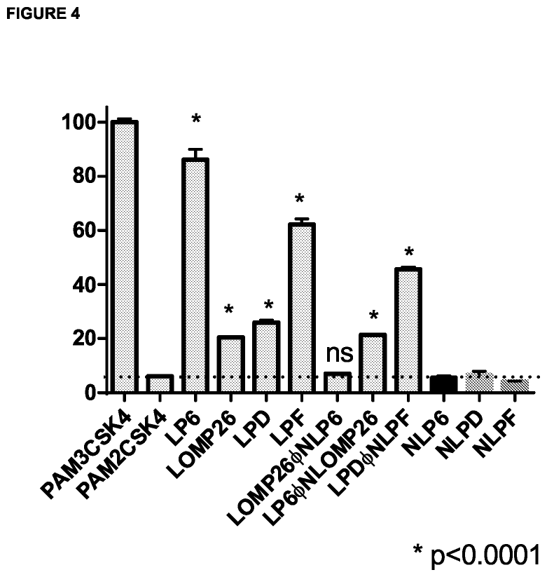

FIG. 4 shows the level of cell stimulation (%) by the various peptides along with positive control PAM3CSK4 (trilipidated peptide) used as 100% stimulation, and negative control PAM2CSK4 (dilipidated peptide). Testing was performed at 3 different concentrations of lipoproteins (10 μg, 1 μg and 0.1 μg of protein). Data shown are at 1 μg. By this method it was estimated that lipidated P6 (LP6) is 86% triacyl lipoprotein, LOMP26 is 21% triacyl, LPD is 26% triacyl, LPF is 62% triacyl, LP6ϕNLOMP26 is 21% triacyl, LPNNLPF is 46% triacyl compared to positive control of Pam3CSK4. LOMP26ϕNLP6 showed no triacyl stimulation, All nonlipidated proteins showed no stimulation (negative control).

The type of signal sequence used to express the fusion proteins was also evaluated to determine if there was any difference in the type or level of acylation on the proteins as a result of the signal sequence. Signal sequences were used to make the constructs and tested in the HEK-BLUE™ hTLR2-TLR1 cells. The different constructs are grown and purified under the same conditions. In all constructs the signal sequence is cleaved at the C residue and the lipid moiety is attached. Sequence of different signals used: Natural P6 signal sequence SS1:MNKFVKSLLVAGSVAALAAC . . . (SEQ ID NO: 36); E. coli natural signal sequence of Pal protein SS2:MQLNKVLKGLMIALPVMAIAAC . . . (SEQ ID NO: 37); H. influenzae signal sequence of P4 protein, SSP4: MKTTLKMTALAALSAFVLAGC . . . (SEQ ID NO: 38).

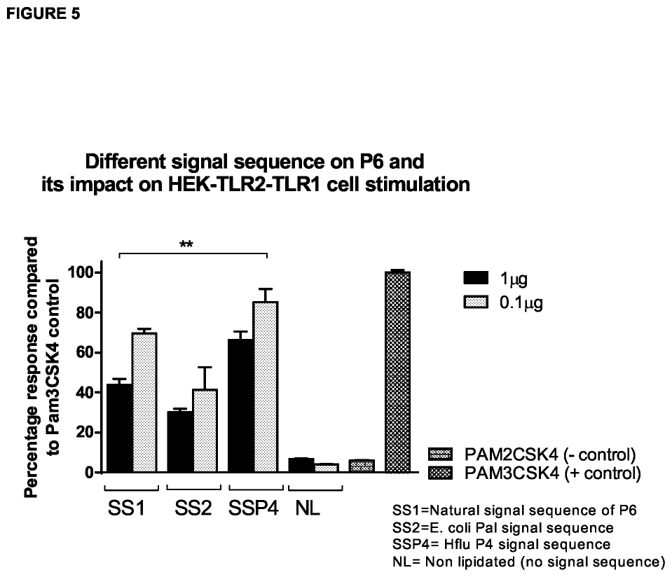

The data in FIG. 5 show that the signal sequence impacts the amount of triacyl lipidation. Constructs using the P4 signal sequence have approximately the same triacyl lipidation levels as those using the P6 natural signal sequence.

### Example 13

### Characterization of Lipidated Proteins

To characterize protein lipidation, mass spectrometry was carried out on LOMP26, LP6 and fusion protein LOMP26P6. DDA runs with full scan (same as previous runs in Example 3 with new gradient, top 10 MS1 precursors are fragmented further) and PRM (parallel reaction monitoring) analysis for selected tripalm precursor m/zs are carried out. Readouts detected triacylated modification, diacylglycerol modification on cysteine and palmitoyl modification on N-terminal of cysteine.

Di and triacyl lipidated moieties may be present in different peaks in mass spectrometry analysis representing variation in lipid tail carbon length and bond number (1 or cyclo). The various compositions presented in the Table show analysis with name modification T1 or D1, modification TA or DA etc.

| Triacyls | Modifications | Diacyls | Modifications |
|---|---|---|---|
| T1 | C16:0, C16:0, C16:1 | D1 | C16:0, C16:1 |
| T2 | C16:0, C16:0, C17:cyclo | D2 | C16:0, C17:cyclo |
| T3 | C16:0, C16:0, C18:1 | D3 | C16:0, C18:1 |
| TA | C16:0, C14:0, C16:1 | DA | C14:0, C16:1 |
| TA′ | C16:0, C16:1, C16:1 | DA′ | C16:1, C16:1 |
| TB | C16:0, C15:0, C16:1 | DB | C15:0, C16:1 |
| TB′ | C16:0, C14:0, C16:0 | DB′ | C14:0, C16:0 |
| TC | C16:0, C16:1, C18:1 | DC | C16:1, C18:1 |
| TD | C16:0, C18:1, C18:1 | DD | C18:1, C18:1 |
| TE | C16:0, C16:0, C19:cyclo | DE | C16:0, C19:cyclo |

Next it was tested whether the media used to culture cells producing the lipidated protein affected the type and amount of lipidation. Cells were grown in 2XYT-broth vs. Minimal media (M9) and levels of lipidation measured. 2XYT broth contains media with 2× as much yeast extract as usual LB media. M9 minimal media contains a minimal salts formulation and nitrogen source (see e.g., ThermoFisher Scientific catalog). Low lipidation yields were observed when 2XYT broth was used, while there was an improved yield of diacyl and triacyl lipoproteins using minimal media (M9). Lipidated proteins were extracted from the membrane using 1% Triton and 1% Zwitterion detergent and purified by Ni-column chromatography and characterized by SDS-PAGE along with Western blot for confirmation. Mass spectrometry comparison of L-OMP26 grown in 2XYT and M9 media showed that the height and magnitude of the lipoprotein peak correlates with the quantity of the specific diacyl or triacyl lipoprotein.

Results demonstrated that there is significantly more diacyl and/or triacyl products when L-OMP26 was grown in minimal media (M9) compared to 2XYT. Additionally, minimal media grown L-OMP26 showed triacylation whereas 2XYT grown L-OMP26 produced no triacylated product. A higher PSM was observed for the diacyl peak grown in minimal media compared to 2XYT media. [PSM is peptide spectrum match, which refers to how many matches between theoretical spectra (generated in-silico based on protein sequence) and the actual spectra from the sample. More matches indicate more abundance of the target peptide].

### Example 14

### Protection by Hi Fusion Proteins in Inoculated Mice

Two models of Hi infection were used to evaluate the effects of lipidated protein vaccines on infection and colonization of animals. In the first model (Model A), mice were immunized D0, D7 and D21 with different lipidated Hi protein constructs. After 3 doses of vaccine, on day 35, mice were infected with PR8 influenza virus as described in a standard model of NTHi acute otitis media (AOM) (Michel et al, J Med Microbiol 2018 67(10):1527-1532) and one week later mice were challenged intranasally with 10 μl of NTHi strain 575 or 86-028NP given to each nostril (Strain 575 has been characterized for surface expression of target proteins and whole genome sequenced). Three days after infection, ear lavage, ear bullae, nasal wash (NW) and blood were harvested. Measurement of NTHi bacteria in NW and middle ear samples were enumerated by plating on chocolate plates.

In a second animal model (Model B), mice were immunized on DO, D7 and D21 with different lipidated Hi protein constructs. On day 35, after 3 doses of vaccine, NTHi inoculation was given to establish colonization, then flu X31 influenza virus (10³ ED50) was administered. Strain X31 of influenza is known to induce a milder infection compared to the previous strain used. Time between NTHi inoculation (10⁶ cfu per mice) and influenza inoculation was 6 hours. Five days after infection, ear lavage, ear bullae, nasal wash (NW) and blood were harvested.

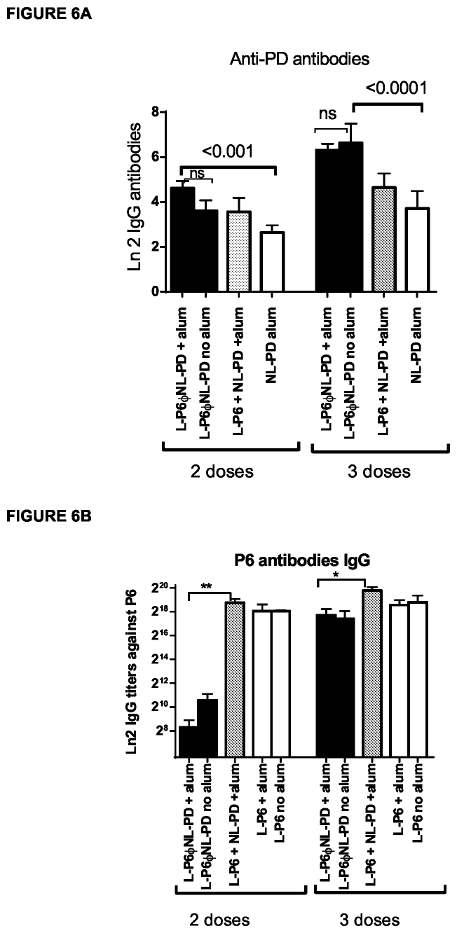

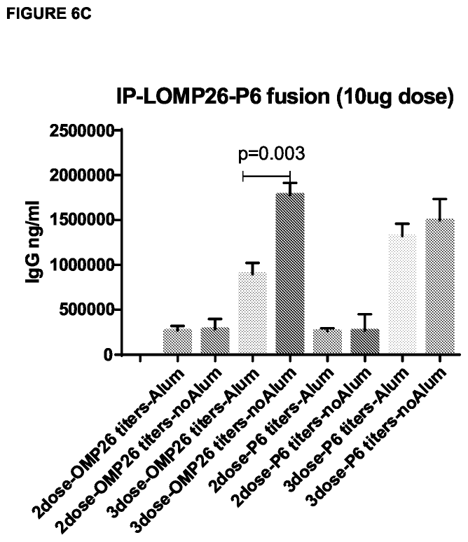

Immunogenicity of lipidated proteins was analyzed (Model B). Alum hydroxide was used as adjuvant. N=6 mice per group. Vaccine was administered intraperitoneally (IP). Molar amount was considered during immunization, and fusion protein was administered at 10 μg, PD at 7 μg and P6 at 3 μg protein per mouse for IP injection. Results are shown in FIG. 6.

A trans effect was observed for protein D responses when non-lipidated protein D fused with lipidated P6 was administered. Trans effect refers to enhanced immunogenicity of the non-lipidated component of the vaccine construct when fused to a lipidated protein in the vaccine. After 3 doses, more than 10 fold increase in antibodies was measured for PD in L-P6φNL-PD fusion without Alum compared to NL-PD with Alum.

It was observed that 3 doses vs. 2 increases immunogenicity. Additionally, fusion may reduce induced lipidated protein antibody response. For example, for L-P6φNL-PD fusion, there was a 2 fold decrease in P6 antibodies observed compared to when L-P6 proteins were administered alone.

Additionally, protein D immunogenicity was tested for lipidated fusion construct in the presence or absence of Alum and no difference in antibody level was measured. For P6 antibodies, L-P6φNL-PD fusion and L-P6 vaccine with/without Alum showed no difference after 2 and 3 doses.

In another experiment, fusions comprising LOMP26 were administered, 10 μg protein dose/mouse were given of proteins set out in FIG. 6C. Alum hydroxide was used as adjuvant, N=4 mice per group, via intraperitoneal (IP) injection. FIG. 6C shows that L-OMP26φNL-P6 fusion produces a significant increase in antibody against OMP26 when lipidated proteins are given without alum compared to when protein was constituted with alum hydoxide. Consistent with previous results, 3 doses of vaccine increases antibody levels compared to 2 doses. Further, alum reduced OMP26 antibody levels, but had no unfavorable effect on P6 antibody levels.

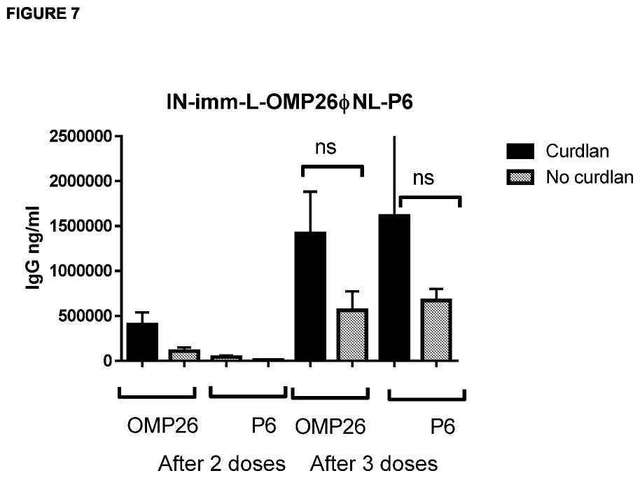

Mice were immunized with OMP26 or P6 (10 μg protein dose/mouse) with or without curdlan adjuvant (N=3-4 mice per group). Results are shown in FIG. 7. Intranasal (IN) immunization with fusion protein L-OMP26φNL-P6 induces significant antibody response, with 3 doses of vaccine increasing antibody levels over 2 doses. There also is a improvement for IN immunization for lipidated protein constructs when adjuvant is used.

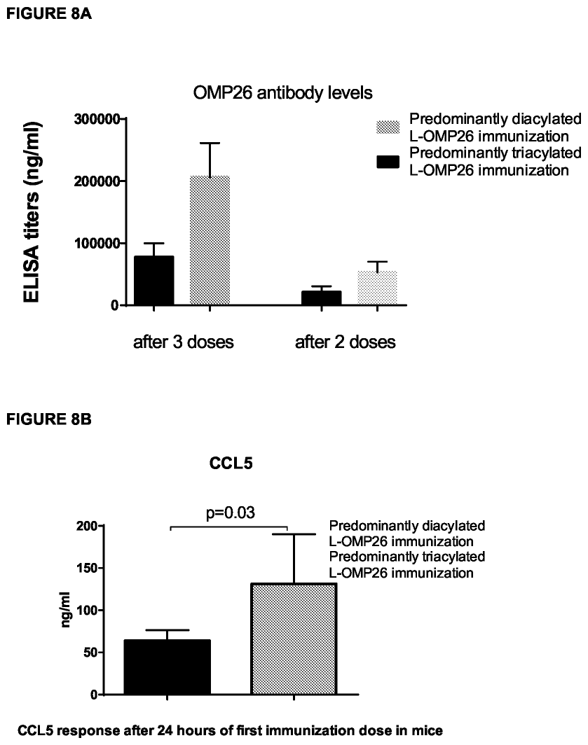

Levels of lipidation were analyzed with respect to immunogenicity. FIG. 8A shows that diacyl moieties produce more antibodies compared to triacyl lipid moieties. L-OMP26 predominantly containing triacyl moieties showed less antibody response compared to diacyl-LOMP26. Diacylation and triacylation status of L-OMP26 is confirmed by Mass-spectrometry based on peak ratio showing 10-fold higher trilipidation (in figure black bar).

Immunogenicity markers were also analyzed. After L-OMP26 immunization without alum 1^{st}, 2^{nd} and 3^{rd} dose, no difference in inflammatory markers IL-8, IL-6, IL-17, or TNF-a was measured, comparing triacyl predominant vs. diacyl predominant compositions, consistent with very low reactogenicity of both constructs. CLL5 cytokines, a surrogate marker of inflammation, was analyzed, and no significant difference in CLL5 24 hours after vaccination found at 2^{nd} and 3^{rd} doses (FIG. 8B) was observed.

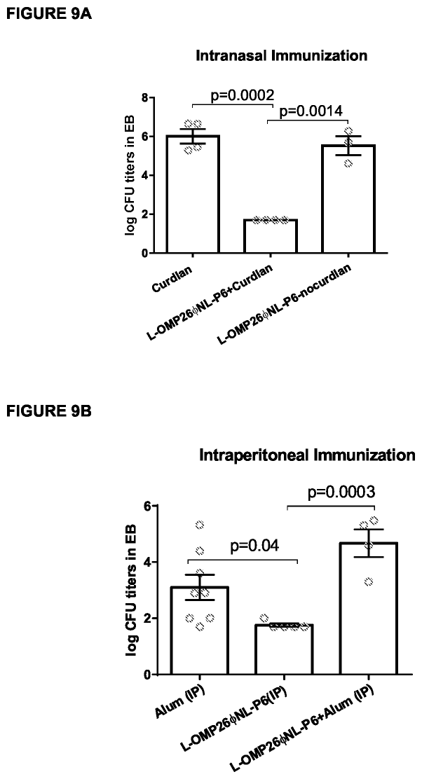

The effects of vaccination on colonization after intranasal immunization was examined. Mice were inoculated with L-OMP26φNL-P6 with or without curdlan adjuvant as in Model B. Results are shown in FIG. 9A, showing protection from ear infection with intranasal immunization with fusion protein L-OMP26φNL-P6. L-OMP26φNL-P6 fusion provides significant protection against NTHi ear infection (FIG. 9B). However, alum hydroxide in the composition appeared to reduce protection from ear infection.

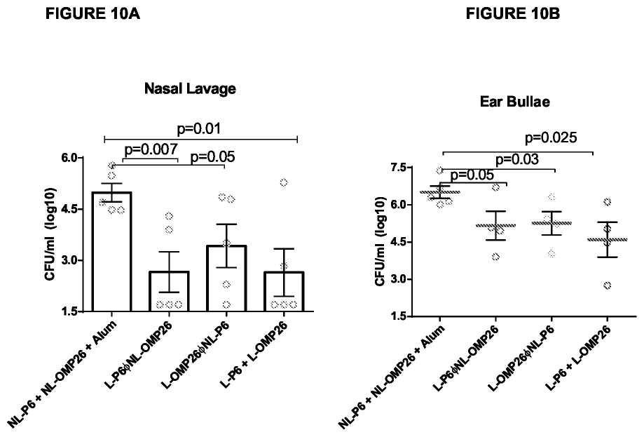

Additional fusion protein constructs were used in Model B immunization: NLOMP26+NLP6, LOMP26NLP6, LP6NLOMP26, and LP6 +LOMP26. Lipidated P6 combined with lipidated OMP26 without alum and lipidated P6 fused to non-lipidated OMP26 protects against nasopharyngeal colonization compared to non-lipidated P6 combined with non-lipidated OMP26 with alum hydroxide as evidenced by nasal lavage and ear bulla colonization levels. Lipidated LP6 combined with lipidated LOMP26 without alum protects against ear infection compared to non-lipidated P6 combined with non lipidated OMP26 with Alum Hydroxide (FIG. 10A).

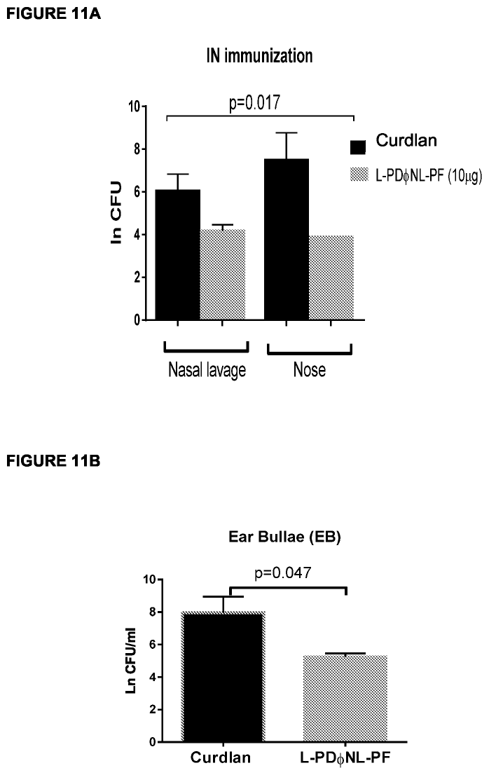

L-PD4NL-PF fusion shows protection in preventing colonization of NTHi after IN immunization. FIG. 11A, 11B. Balb c mice (n=4/group) were challenged with 14-02-575 (in house clinical strain of NTHi), 10 μg of protein was used per dose and 3 doses were given. In the colonization model, CFU were detectable in ear 12 hours after challenge and a significant difference in colonization levels was observed with fusion LPDϕPF.

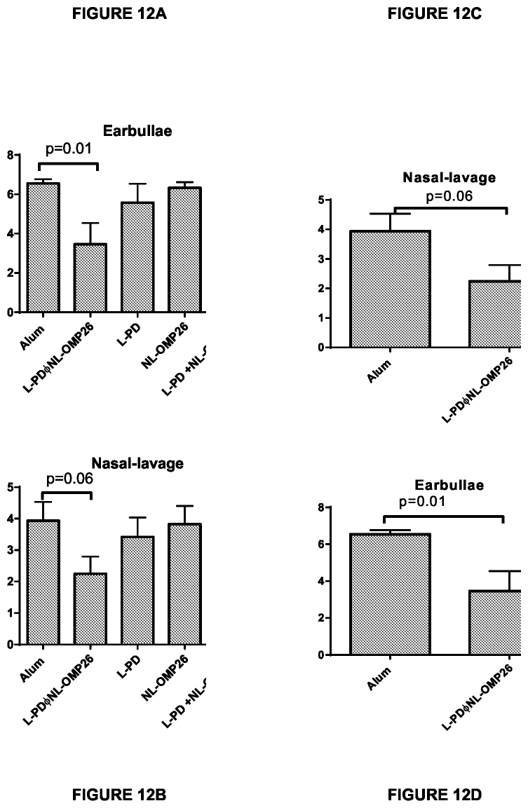

Intramuscular immunization was also tested. For L-PD4NL-OMP26 fusion via IM immunization (10 μg/dose), protection was observed in ear infection and nasopharyngeal colonization (FIG. 12A, 12B). L-PD and NL-OMP26 administered separately did not show protection. In a separate experiment, different combinations of Hi proteins were used in Model B, with 10 μg/dose, for 3 doses (n=5-6 mice/group. FIGS. 12C and 12D illustrate that L-PDϕNL-OMP26 fusion protein administered by IM immunization (10 μg protein/dose), protected against both nasopharyngeal colonization and ear infection.

Numerous modifications and variations of the invention as set forth in the above illustrative examples are expected to occur to those skilled in the art. Consequently only such limitations as appear in the appended claims should be placed on the invention.

1. A fusion protein comprising all or part of two or more Haemophilus influenzae (Hi) proteins selected from the group consisting of Omp26, P6, PD and PF, wherein at least one of the Hi proteins is lipidated.

2. The fusion protein of claim 1, comprising Omp26 and P6, wherein the Omp 26 protein or fragment thereof is lipidated, or wherein the P6 protein or fragment thereof is lipidated.

3. (canceled)

4. The fusion protein of claim 1, wherein the at least one Hi protein comprises a lipid moiety selected from C18, C16, C14, C12, or C10 fatty acid.

5. The fusion protein of claim 1, wherein the at least one Hi protein comprises a lipid moiety selected from i) a diacyl and/or a triacyl fatty acid; and/or, ii) an N-acylated or O-acylated fatty acid.

6-7. (canceled)

8. The fusion protein of claim 1 comprising lipidated Omp26 and non-lipidated P6, wherein the lipid comprises a C16 fatty acid or comprising lipidated P6 and non-lipidated Omp26, wherein the lipid is a C16 fatty acid.

9. (canceled)

10. The fusion protein of claim 1, wherein the fusion protein is selected from the group consisting of LOmp26φP6 (SEQ ID NO: 31), LP6φOmp26 (SEQ ID NO: 29), L-P6φNL-PD (SEQ ID NO: 39), L-PDφNL-PF (SEQ ID NO: 40), L-PDφNL-P6 (SEQ ID NO: 41), L-P6φNL-PD (SEQ ID NO: 42), L-OMP26φNL-PD (SEQ ID NO: 43), L-PDφNL-OMP26 (SEQ ID NO: 44), or L-PFφNL-P6 (SEQ ID NO: 45).

11. The fusion protein of claim 8, wherein Omp26 comprises all or part of the amino acid sequence set out in SEQ ID NO: 2 and/or wherein P6 comprises all or part of the amino acid sequence set out in SEQ ID NO: 4.

12. (canceled)

13. The fusion protein of claim 1, wherein the at least one Hi protein comprises a lipid moiety at the N terminus of the protein.

14. The fusion protein of claim 1, wherein the Hi proteins are linked by a peptide linker.

15. (canceled)

16. The fusion protein of claim 1 comprising the amino acid sequence set out in SEQ ID NO: 29 or 31.

17. The fusion protein of claim 1 further comprising a pharmaceutically acceptable carrier or excipient.

18. A vaccine comprising the fusion protein of claim 1.

19. The vaccine of claim 18, wherein the fusion protein in the vaccine elicits an antibody response against the lipidated Hi protein and at least one non-lipidated Hi proteins.

20. The vaccine of claim 18, wherein at least 10% of the fusion proteins in the vaccine comprises a diacyl fatty acid and/or wherein at least 10% of the fusion proteins in the vaccine comprises a triacyl fatty acid.

21. (canceled)

22. A immunogenic composition comprising the fusion protein of claim 1.

23. The immunogenic composition of claim 22, wherein at least 10% of the fusion proteins in the immunogenic composition comprises a diacyl fatty acid and/or wherein at least 10% of the fusion proteins in the vaccine comprises a triacyl fatty acid.

24. (canceled)

25. A method of treating or preventing a disorder associated with Haemophilus influenza (Hi) infection in a subject comprising administering the vaccine of claim 18 to the subject.

26. The method of claim 25, wherein the disorder associated with Hi infection is otitis media, bronchitis, pneumonia, sinusitis, septicemia, endocarditis, epiglottitis, septic arthritis, meningitis, postpartum and neonatal infections, postpartum and neonatal sepsis, acute and chronic salpingitis, epiglottis, pericarditis, cellulitis, osteomyelitis, endocarditis, cholecystitis, intraabdominal infections, urinary tract infection, mastoiditis, aortic graft infection, conjunctitivitis, Brazilian purpuric fever, occult bacteremia and exacerbation of underlying lung diseases such as chronic bronchitis, bronchietasis or cystic fibrosis, chronic obstructive pulmonary disorder (COPD), and acute exacerbations of COPD (AECOPD).

27. The method of claim 25, wherein the vaccine or immunogenic composition is administered orally, intramuscularly, intranasally, or subcutaneously.

28. (canceled)

29. The method of claim 25, wherein the vaccine or immunogenic compositions comprises an LOmp26φP6 or LP6ϕOmp26.

30. A method of making a fusion protein comprising all or part of two or more Haemophilus influenzae (Hi) proteins selected from the group consisting of Omp26, P6, PD and PF, wherein at least one of the Hi proteins is lipidated, the method comprising:
i) inserting a nucleic acid encoding a lipid moiety signal sequence region upstream of a first nucleic acid encoding all or part of a Haemophilus influenzae (Hi) protein Omp26, P6, PD or PF in a plasmid vector;
ii) inserting a second nucleic acid encoding all or part of a Haemophilus influenzae (Hi) protein Omp26, P6, PD or PF in the plasmid vector;
iii) optionally inserting a third or additional nucleic acid sequences encoding one or more additional Hi proteins or fragment thereof selected from the group consisting of Omp26, P6, PD and PF;
iii) transfecting the plasmid vector into a host cell capable of expressing the nucleic acid molecules; and
iv) purifying the fusion protein expressed by the plasmid.

31. A method of making a fusion protein comprising all or part of two or more Haemophilus influenzae (Hi) proteins selected from the group consisting of Omp26, P6, PD and PF, wherein at least one of the Hi proteins is lipidated, the method comprising:
i) providing a nucleic acid encoding a lipid moiety signal sequence region;
ii) providing a first nucleic acid encoding all or part of a Hi protein Omp26, P6, PD or PF;
iii) providing a second nucleic acid encoding all or part of a Hi protein Omp26, P6, PD or PF, wherein the second nucleic acid encodes a different Hi protein from i);
iv) optionally providing a third or additional nucleic acid sequences encoding one or more additional proteins or fragment thereof selected from the group consisting of Omp26, P6, PD and PF;
v) inserting the nucleic acid sequences i-iv) into a plasmid vector capable of expressing the nucleic acids;
vi) transfecting the plasmid vector of iv) into a host cell capable of expressing the nucleic acid molecules and expressing the fusion protein; and
vii) purifying the recombinant fusion protein expressed in the host cell.

32. The method of claim 30, wherein the nucleic acid encoding the lipid moiety signal sequence is inserted upstream of the first nucleic acid molecule in the plasmid vector and the second nucleic acid, and optional third and additional nucleic acids, are inserted downstream of the first nucleic acid in the plasmid vector; or
wherein the nucleic acid encoding the lipid moiety signal sequence is inserted upstream of the second nucleic acid molecule in the plasmid vector and the first nucleic acid is inserted upstream of the lipid moiety signal sequence, and optional third or additional nucleic acids are inserted downstream of the second nucleic acid in the plasmid vector.

33-35. (canceled)

36. The method of claim 30, further comprising a nucleic acid encoding a peptide linker sequence inserted between the first and second nucleic acids, and/or between the second and optional third and additional nucleic acids.

37. (canceled)

38. The method of claim 30, further comprising an additional nucleic acid encoding a lipid moiety signal sequence upstream of a nucleic acid encoding a Hi protein or fragment thereof selected from the group consisting of Omp26, P6, PD and PF.

39. The method of claim 30, wherein the first nucleic acid encodes for a P6 protein or fragment thereof and the second nucleic acid encodes for a Omp26 protein or fragment thereof, or wherein the first nucleic acid encodes for a Omp26 protein or fragment thereof and the second nucleic acid encodes for a P6 protein or fragment thereof.

40-54. (canceled)

55. The fusion protein of claim 1, wherein the at least one of the Hi proteins that is lipidated is a non-naturally lipidated protein.


SEQUENCE LISTING (45 sequences)

SEQ ID NO: 1 - DNA, 552 bp, Artificial Sequence
  Synthetic
atggaggaaaagatcgcgttcattaacgcgggttacatctttcaacatcacccggaccgtcaagcggtggcggacaaactggatgcggagttcaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagttcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaactcgagcaccaccaccaccaccactga

SEQ ID NO: 2 - protein, 184 aa, Artificial Sequence
  Synthetic
MEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKKLEHHHHHH

SEQ ID NO: 3 - DNA, 429 bp, Artificial Sequence
  Synthetic
atgagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatctcgagcaccaccaccaccaccactga

SEQ ID NO: 4 - protein, 143 aa, Artificial Sequence
  Synthetic
MSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYKLEHHHHHH

SEQ ID NO: 5 - DNA, 783 bp, Artificial Sequence
  Synthetic
atgggtagccaccaaatgaagagcgagggtcacgcgaacatgcaactgcagcaacaggcggtgctgggtctgaactggatgcaagacagcggcgagtacaaagcgctggcgtaccaggcgtataacgcggcgaaggtggcgttcgatcacgcgaaagttgcgaagggcaagaaaaaggcggtggttgcggacctggatgaaaccatgctggacaacagcccgtatgcgggttggcaagtgcagaacaacaaaccgtttgacggcaaggattggacccgttgggttgatgcgcgtcaaagccgtgcggtgccgggtgcggttgagttcaacaactacgtgaacagccacaacggcaaagtgttttatgttaccaaccgtaaagacagcaccgaaaagagcggcaccatcgacgatatgaagcgtctgggtttcaacggcgttgaggaaagcgcgttttacctgaaaaaggacaaaagcgcgaaggcggcgcgttttgcggagatcgaaaaacaaggttacgagattgtgctgtatgttggcgataacctggacgatttcggtaacaccgtgtatggcaagctgaacgcggaccgtcgtgcgtttgttgatcaaaaccagggtaaattcggcaagacctttattatgctgccgcaggcgaactatggtggctgggagggtggcctggcggaaggttattttaaaaaggatacccaaggccagatcaaagcgcgcctggacgcggttcaagcgtgggacggcaagcatcatcaccatcatcactaa

SEQ ID NO: 6 - protein, 280 aa, Artificial Sequence
  Synthetic
MKTTLKMTALAALSAFVLAGCGSHQMKSEGHANMQLQQQAVLGLNWMQDSGEYKALAYQAYNAAKVAFDHAKVAKGKKKAVVADLDETMLDNSPYAGWQVQNNKPFDGKDWTRWVDARQSRAVPGAVEFNNYVNSHNGKVFYVTNRKDSTEKSGTIDDMKRLGFNGVEESAFYLKKDKSAKAARFAEIEKQGYEIVLYVGDNLDDFGNTVYGKLNADRRAFVDQNQGKFGKTFIMLPQANYGGWEGGLAEGYFKKDTQGQIKARLDAVQAWDGKHHHHHH

SEQ ID NO: 7 - DNA, 1065 bp, Artificial Sequence
  Synthetic
atgagcagccacagcagcaacatggcgaacacccagatgaagagcgataaaatcattattgcgcaccgtggcgcgagcggttacctgccggagcacaccctggaaagcaaagcgctggcgtttgcgcagcacgcggattatctggagcaagacctggcgatgaccaaagacggccgtctggtggttattcacgaccacttcctggatggtctgaccgacgtggcgaagaaatttccgcaccgtcaccgtaaggatggccgttactatgttattgacttcaccctgaaagaaatccaaagcctggaaatgaccgagaactttgaaaccaaggatggtaaacaggcgcaagtgtacccgaaccgtttcccgctgtggaagagccacttccgtattcacacctttgaggacgaaattgagtttatccagggcctggagaagagcaccggcaagaaagttggtatttatccggaaatcaaagcgccgtggttccaccaccaaaacggcaaggacatcgcggcggagaccctgaaagtgctgaagaaatacggttatgataagaaaaccgacatggtttacctgcagaccttcgattttaacgagctgaagcgtattaaaaccgaactgctgccgcagatgggtatggacctgaagctggtgcaactgatcgcgtacaccgattggaaagagacccaagaaaaggacccgaaaggctattgggttaactacaactatgattggatgtttaagccgggtgcgatggcggaagtggttaaatacgcggacggtgtgggcccgggttggtatatgctggttaacaaagaggagagcaaaccggataacattgtgtacaccccgctggttaaggagctggcgcagtacaacgtggaactgcacccgtataccgttcgtaaagacgcgctgccggagttctttaccgatgtgaaccaaatgtatgacaccctgctgaacaaaagcggcgcgaccggtgtgttcaccgactttccggataccggcgttgaatttctgaagggtatcaaactcgagcaccaccaccaccaccactga

SEQ ID NO: 8 - protein, 354 aa, Artificial Sequence
  Synthetic
MSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIKLEHHHHHH

SEQ ID NO: 9 - DNA, 849 bp, Artificial Sequence
  Synthetic
agcagcggcaagttcaaagtggttaccacctttaccgtgatccaggacattgcgcaaaacgttgcgggtgatgcggcgaccgtggagagcatcaccaagccgggtgcggaaattcacgagtacgaaccgaccccgaaggacatcgttaaagcgcagagcgcggatctgattctgtggaacggtctgaacctggagcgttggtttgaacgtttctttcagaacgtgaaggacaaaccggcggtggttgtgaccgagggtatccaaccgctgagcatttacgaaggcccgtataaagatgcgccgaacccgcacgcgtggatgagcccgagcaacgcgctgatctacattgagaacatcaagaacgcgctggttaaatatgacccgcagaacgcggcggtgtacgagaagaacgcggcggactatgcgcagaagatcaaacaactggatgaaccgctgcgtgcgaaactggcgcagattccggaggcgcaacgttggctggttaccagcgaaggcgcgttcagctacctggcgaaggactataacctgaaagagggttacctgtggccgatcaacgcggaacagcaaggcaccccgcagcaagttcgtaaggtgatcgatctggttcgtaaaaacaacattccggttgtgtttagcgagagcaccattagcgcgaagccggcgcagcaagtggcgaaggaaagcggtgcgaaatacggtggcgttctgtatgtggacagcctgagcgcgaaaaacggcccggttccgacctatatcgatctgctgaacgttaccgtgagcaccattgtgaagggtttcggcaaactcgagcaccaccaccaccaccactga

SEQ ID NO: 10 - protein, 282 aa, Artificial Sequence
  Synthetic
SSGKFKVVTTFTVIQDIAQNVAGDAATVESITKPGAEIHEYEPTPKDIVKAQSADLILWNGLNLERWFERFFQNVKDKPAVVVTEGIQPLSIYEGPYKDAPNPHAWMSPSNALIYIENIKNALVKYDPQNAAVYEKNAADYAQKIKQLDEPLRAKLAQIPEAQRWLVTSEGAFSYLAKDYNLKEGYLWPINAEQQGTPQQVRKVIDLVRKNNIPVVFSESTISAKPAQQVAKESGAKYGGVLYVDSLSAKNGPVPTYIDLLNVTVSTIVKGFGKLEHHHHHH

SEQ ID NO: 11 - protein, 20 aa, Artificial Sequence
  Synthetic
MKTTLKMTALAALSAFVLAG

SEQ ID NO: 12 - protein, 6 aa, Artificial Sequence
  Synthetic
GSGGGG

SEQ ID NO: 13 - DNA, 621 bp, Artificial Sequence
  Synthetic
atgaagaccaccctgaaaatgaccgcgctggcggcgctgagcgcgttcgttctggcgggttgcagcagcggcgaggaaaagatcgcgttcattaacgcgggttacatctttcaacatcacccggaccgtcaagcggtggcggacaaactggatgcggagtttaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagtgcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaactcgagcaccaccaccaccaccactga

SEQ ID NO: 14 - protein, 206 aa, Artificial Sequence
  Synthetic
MKTTLKMTALAALSAFVLAGCSSGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKLEHHHHHH

SEQ ID NO: 15 - DNA, 489 bp, Artificial Sequence
  Synthetic
atgaagaccaccctgaaaatgaccgcgctggcggcgctgagcgcgtttgtgctggcgggttgcagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatctcgagcaccaccaccaccaccactga

SEQ ID NO: 16 - protein, 162 aa, Artificial Sequence
  Synthetic
MKTTLKMTALAALSAFVLAGCSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYLEHHHHHH

SEQ ID NO: 17 - DNA, 1125 bp, Artificial Sequence
  Synthetic
atgaagaccaccctgaaaatgaccgcgctggcggcgctgagcgcgtttgtgctggcgggttgcagcagccacagcagcaacatggcgaacacccagatgaagagcgataaaatcattattgcgcaccgtggcgcgagcggttacctgccggagcacaccctggaaagcaaagcgctggcgtttgcgcagcacgcggattatctggagcaagacctggcgatgaccaaagacggccgtctggtggttattcacgaccacttcctggatggtctgaccgacgtggcgaagaaatttccgcaccgtcaccgtaaggatggccgttactatgttattgacttcaccctgaaagaaatccaaagcctggaaatgaccgagaactttgaaaccaaggatggtaaacaggcgcaagtgtacccgaaccgtttcccgctgtggaagagccacttccgtattcacacctttgaggacgaaattgagtttatccagggcctggagaagagcaccggcaagaaagttggtatttatccggaaatcaaagcgccgtggttccaccaccaaaacggcaaggacatcgcggcggagaccctgaaagtgctgaagaaatacggttatgataagaaaaccgacatggtttacctgcagaccttcgattttaacgagctgaagcgtattaaaaccgaactgctgccgcagatgggtatggacctgaagctggtgcaactgatcgcgtacaccgattggaaagagacccaagaaaaggacccgaaaggctattgggttaactacaactatgattggatgtttaagccgggtgcgatggcggaagtggttaaatacgcggacggtgtgggcccgggttggtatatgctggttaacaaagaggagagcaaaccggataacattgtgtacaccccgctggttaaggagctggcgcagtacaacgtggaactgcacccgtataccgttcgtaaagacgcgctgccggagttctttaccgatgttaaccaaatgtatgacaccctgctgaacaaaagcggcgcgaccggtgtgttcaccgactttccggataccggcgttgaattcctgaagggtatcaaactcgagcaccaccaccaccaccactga

SEQ ID NO: 18 - protein, 374 aa, Artificial Sequence
  Synthetic
MKTTLKMTALAALSAFVLAGCSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIKLEHHHHHH

SEQ ID NO: 19 - DNA, 843 bp, Artificial Sequence
  Synthetic
atgaagaccaccctgaaaatgaccgcgctggcggcgctgagcgcgttcgttctggcgggttgcggtagccaccaaatgaagagcgagggtcacgcgaacatgcaactgcagcaacaggcggtgctgggtctgaactggatgcaagacagcggcgagtacaaagcgctggcgtaccaggcgtataacgcggcgaaggtggcgttcgatcacgcgaaagttgcgaagggcaagaaaaaggcggtggttgcggacctggatgaaaccatgctggacaacagcccgtatgcgggttggcaagtgcagaacaacaaaccgtttgacggcaaggattggacccgttgggttgatgcgcgtcaaagccgtgcggtgccgggtgcggttgagttcaacaactacgtgaacagccacaacggcaaagtgttttatgttaccaaccgtaaagacagcaccgaaaagagcggcaccatcgacgatatgaagcgtctgggtttcaacggcgttgaggaaagcgcgttttacctgaaaaaggacaaaagcgcgaaggcggcgcgttttgcggagatcgaaaaacaaggttacgagattgtgctgtatgttggcgataacctggacgatttcggtaacaccgtgtatggcaagctgaacgcggaccgtcgtgcgtttgttgatcaaaaccagggtaaattcggcaagacctttattatgctgccgcaggcgaactatggtggctgggagggtggcctggcggaaggttattttaaaaaggatacccaaggccagatcaaagcgcgcctggacgcggttcaagcgtgggacggcaagcatcatcaccatcatcactaa

SEQ ID NO: 20 - protein, 280 aa, Artificial Sequence; identical to SEQ ID NO: 6
  Synthetic

SEQ ID NO: 21 - DNA, 30 bp, Artificial Sequence
  Synthetic
gatatacatatgaagaccaccctgaaaatg

SEQ ID NO: 22 - DNA, 28 bp, Artificial Sequence
  Synthetic
ctatatctcgagtttcttttcttgcact

SEQ ID NO: 23 - DNA, 30 bp, Artificial Sequence
  Synthetic
gatatacatatggaggaaaagatcgcgttc

SEQ ID NO: 24 - DNA, 28 bp, Artificial Sequence
  Synthetic
ctatatctcgagatacgccagaaccgca

SEQ ID NO: 25 - DNA, 31 bp, Artificial Sequence
  Synthetic
gatatacatatgagcagcagcaacaacgatg

SEQ ID NO: 26 - DNA, 29 bp, Artificial Sequence
  Synthetic
ctatatctcgagtttgatacccttcagaa

SEQ ID NO: 27 - DNA, 30 bp, Artificial Sequence
  Synthetic
gatatacatatgagcagccacagcagcaac

SEQ ID NO: 28 - DNA, 1029 bp, Artificial Sequence
  Synthetic
atgaagaccaccctgaaaatgaccgcgctggcggcgctgagcgcgtttgtgctggcgggttgcagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatggtagtggcggcgggggagaggaaaagatcgcgttcattaacgcgggttacatctttcaacatcacccggaccgtcaagcggtggcggacaaactggatgcggagtttaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagtgcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaactcgagcaccaccaccaccaccactga

SEQ ID NO: 29 - protein, 342 aa, Artificial Sequence
  Synthetic
MKTTLKMTALAALSAFVLAGCSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYGSGGGGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKLEHHHHHH

SEQ ID NO: 30 - DNA, 1038 bp, Artificial Sequence
  Synthetic
atgaagaccaccctgaaaatgaccgcgctggcggcgctgagcgcgttcgttctggcgggttgcagcagcggcgaggaaaagatcgcgttcattaacgcgggttacatctttcaacatcacccggaccgtcaagcggtggcggacaaactggatgcggagtttaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagtgcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaaggtagtggcggcgggggaagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatctcgagcaccaccaccaccaccactga

SEQ ID NO: 31 - protein, 345 aa, Artificial Sequence
  Synthetic
MKTTLKMTALAALSAFVLAGCSSGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKGSGGGGSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYLEHHHHHH

SEQ ID NO: 32 - DNA, 969 bp, Artificial Sequence
  Synthetic
atgagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatggtagtggcggcgggggagaggaaaagatcgcgttcattaacgcgggttacatctttcaacatcacccggaccgtcaagcggtggcggacaaactggatgcggagtttaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagtgcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaactcgagcaccaccaccaccaccactga

SEQ ID NO: 33 - protein, 322 aa, Artificial Sequence
  Synthetic
MSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYGSGGGGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKLEHHHHHH

SEQ ID NO: 34 - DNA, 969 bp, Artificial Sequence
  Synthetic
atggaggaaaagatcgcgttcattaacgcgggttacatctttcaacatcacccggaccgtcaagcggtggcggacaaactggatgcggagtttaagccggttgcggaaaaactggcggcgagcaagaaagaggtggacgataagattgcggcggcgcgtaagaaagtggaggcgaaggttgcggcgctggaaaaagacgcgccgcgtctgcgtcaggcggatatccagaagcgtcaagaggaaattaacaaactgggtgcggcggaggacgcggaactgcaaaaactgatgcaggagcaagataagaaagtgcaggagttccaggcgcaaaacgaaaagcgtcaagcggaggaacgtggcaaactgctgaacagcatccagaccgcgaccaacaacctggcgaaggcgaaaggttacacctatgttctggacgcgaacagcgtggtttttgcggtggaaggcaaggatatcaccgaggaagttctgaagagcattccggcgagcgagaaagtgcaagaaaagaaaggtagtggcggcgggggaagcagcagcaacaacgatgcggcgggtaacggtgcggcgcagacctttggtggctacagcgtggcggatctgcagcaacgttacaacaccgtttacttcggtttcgacaagtacgatatcaccggcgaatatgtgcaaattctggacgcgcacgcggcgtatctgaacgcgaccccggcggcgaaggtgctggttgagggtaacaccgatgaacgtggcaccccggagtacaacattgcgctgggtcagcgtcgtgcggatgcggttaagggctatctggcgggtaaaggcgtggatgcgggcaagctgggcaccgttagctacggcgaggaaaaaccggcggtgctgggtcatgatgaggcggcgtacagcaagaaccgtcgtgcggttctggcgtatctcgagcaccaccaccaccaccactga

SEQ ID NO: 35 - protein, 322 aa, Artificial Sequence
  Synthetic
MEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKGSGGGGSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYLEHHHHHH

SEQ ID NO: 36 - protein, 20 aa, Artificial Sequence
  Synthetic
MNKFVKSLLVAGSVAALAAC

SEQ ID NO: 37 - protein, 22 aa, Artificial Sequence
  Synthetic
MQLNKVLKGLMIALPVMAIAAC

SEQ ID NO: 38 - protein, 21 aa, Artificial Sequence
  Synthetic
MKTTLKMTALAALSAFVLAGC

SEQ ID NO: 39 - protein, 505 aa, Artificial Sequence
  Synthetic
MKTTLKMTALAALSAFVLAGCSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAYGSGGGGSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIK

SEQ ID NO: 40 - protein, 642 aa, Artificial Sequence
  Synthetic
MKTTLKMTALAALSAFVLAGCSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIKGSGGGGKFKVVTTFTVIQDIAQNVAGDAATVESIKPGAEIHEYEPTPKDIVKAQSADLILWNGLNLERWFERFFQNVKDKPAVVVTEGIQPLSIYEGPYKDAPNPHAWMSPSNALIYIENIKNALVKYDPQNAAVYEKNAADYAQKIKQLDEPLRAKLAQIPEAQRWLVTSEGAFSYLAKDYNLKEGYLWPINAEQQGTPQQVRKVIDLVRKNNIPVVFSESTISAKPAQQVAKESGAKYGGVLYVDSLSAKNGPVPTYIDLLNVTVSTIVKGFGK

SEQ ID NO: 41 - protein, 505 aa, Artificial Sequence
  Synthetic
MKTTLKMTALAALSAFVLAGCSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIKGSGGGGSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAY

SEQ ID NO: 42 - protein, 505 aa, Artificial Sequence; identical to SEQ ID NO: 39
  Synthetic

SEQ ID NO: 43 - protein, 549 aa, Artificial Sequence
  Synthetic
MKTTLKMTALAALSAFVLAGCSSGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKKGSGGGGSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIK

SEQ ID NO: 44 - protein, 546 aa, Artificial Sequence
  Synthetic
MKTTLKMTALAALSAFVLAGCSSHSSNMANTQMKSDKIIIAHRGASGYLPEHTLESKALAFAQHADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVELHPYTVRKDALPEFFTDVNQMYDTLLNKSGATGVFTDFPDTGVEFLKGIKGSGGGGEEKIAFINAGYIFQHHPDRQAVADKLDAEFKPVAEKLAASKKEVDDKIAAARKKVEAKVAALEKDAPRLRQADIQKRQEEINKLGAAEDAELQKLMQEQDKKVQEFQAQNEKRQAEERGKLLNSIQTATNNLAKAKGYTYVLDANSVVFAVEGKDITEEVLKSIPASEKVQEKK

SEQ ID NO: 45 - protein, 433 aa, Artificial Sequence
  Synthetic
MKTTLKMTALAALSAFVLAGCSSGKFKVVTTFTVIQDIAQNVAGDAATVESITKPGAEIHEYEPTPKDIVKAQSADLILWNGLNLERWFERFFQNVKDKPAVVVTEGIQPLSIYEGPYKDAPNPHAWMSPSNLIYIENIKNALVKYDPQNAAVYEKNAADYAQKIKQLDEPLRAKLAQIPEAQRWLVTSEGAFSYLAKDYNLKEGYLWPINAEQQGTPQQVRKVIDLVRKNNIPVVFSESTISAKPAQQVAKESGAKYGGVLYVDSLSAKNGPVPTYIDLLNVTVSTIVKGFGKGSGGGGSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAY